SEQUENCE LISTING (149 sequences)

SEQ ID NO: 1 - protein, 10 aa, Artificial Sequence
  Delta V1-1 peptide
SFNSYELGSL

SEQ ID NO: 2 - protein, 10 aa, Artificial Sequence
  Delta V1-2 peptide
SFNSYELGSL

SEQ ID NO: 3 - protein, 14 aa, Artificial Sequence
  Delta V1-5 peptide
KAEFWLDLQPQAKV

SEQ ID NO: 4 - protein, 58 aa, Artificial Sequence
  Delta V5 polypeptide
PFRPKVKSPRPYSNFDQEFLNEKARLSYSDKNLIDSMDQSAFAGFSFVNPKFEHLLED

SEQ ID NO: 5 - protein, 10 aa, Artificial Sequence
  Modified Delta V1-1 peptide
TFNSYELGSL

SEQ ID NO: 6 - protein, 10 aa, Artificial Sequence
  Modified Delta V1-1 peptide
AFNSYELGSL

SEQ ID NO: 7 - protein, 10 aa, Artificial Sequence
  Modified Delta V1-1 peptide
SFNSYELGTL

SEQ ID NO: 8 - protein, 10 aa, Artificial Sequence
  Modified Delta V1-1 peptide
TFNSYELGTL

SEQ ID NO: 9 - protein, 10 aa, Artificial Sequence
  Modified Delta V1-1 peptide
SYNSYELGSL

SEQ ID NO: 10 - protein, 10 aa, Artificial Sequence
  Modified Delta V1-1 peptide
SFNSFELGSL

SEQ ID NO: 11 - protein, 9 aa, Artificial Sequence
  Modified Delta V1-1 peptide
SNSYDLGSL

SEQ ID NO: 12 - protein, 10 aa, Artificial Sequence
  Modified Delta V1-1 peptide
SFNSYELPSL

SEQ ID NO: 13 - protein, 10 aa, Artificial Sequence
  Modified Delta V1-1 peptide
SFNSYEIGSV

SEQ ID NO: 14 - protein, 10 aa, Artificial Sequence
  Modified Delta V1-1 peptide
SFNSYEVGSI

SEQ ID NO: 15 - protein, 10 aa, Artificial Sequence
  Modified Delta V1-1 peptide
SFNSYELGSV

SEQ ID NO: 16 - protein, 10 aa, Artificial Sequence
  Modified Delta V1-1 peptide
SFNSYELGSI

SEQ ID NO: 17 - protein, 10 aa, Artificial Sequence
  Modified Delta V1-1 peptide
SFNSYEIGSL

SEQ ID NO: 18 - protein, 10 aa, Artificial Sequence
  Modified Delta V1-1 peptide
SFNSYEVGSL

SEQ ID NO: 19 - protein, 10 aa, Artificial Sequence
  Modified Delta V1-1 peptide
AFNSYELGSL

SEQ ID NO: 20 - protein, 6 aa, Artificial Sequence
  Modified Delta V1-1 peptide
YELGSL

SEQ ID NO: 21 - protein, 6 aa, Artificial Sequence
  Modified Delta V1-1 peptide
YDLGSL

SEQ ID NO: 22 - protein, 6 aa, Artificial Sequence
  Modified Delta V1-1 peptide
FDLGSL

SEQ ID NO: 23 - protein, 6 aa, Artificial Sequence
  Modified Delta V1-1 peptide
YDIGSL

SEQ ID NO: 24 - protein, 4 aa, Artificial Sequence
  Modified Delta V1-1 peptide
IGSL

SEQ ID NO: 25 - protein, 6 aa, Artificial Sequence
  Modified Delta V1-1 peptide
YDVGSL

SEQ ID NO: 26 - protein, 6 aa, Artificial Sequence
  Modified Delta V1-1 peptide
YDLPSL

SEQ ID NO: 27 - protein, 6 aa, Artificial Sequence
  Modified Delta V1-1 peptide
YDLGIL

SEQ ID NO: 28 - protein, 6 aa, Artificial Sequence
  Modified Delta V1-1 peptide
YDLGSI

SEQ ID NO: 29 - protein, 6 aa, Artificial Sequence
  Modified Delta V1-1 peptide
YDLGSV

SEQ ID NO: 30 - protein, 4 aa, Artificial Sequence
  Modified Delta V1-1 peptide
LGSL

SEQ ID NO: 31 - protein, 4 aa, Artificial Sequence
  Modified Delta V1-1 peptide
IGSL

SEQ ID NO: 32 - protein, 4 aa, Artificial Sequence
  Modified Delta V1-1 peptide
VGSL

SEQ ID NO: 33 - protein, 4 aa, Artificial Sequence
  Modified Delta V1-1 peptide
LPSL

SEQ ID NO: 34 - protein, 4 aa, Artificial Sequence
  Modified Delta V1-1 peptide
LGIL

SEQ ID NO: 35 - protein, 4 aa, Artificial Sequence
  Modified Delta V1-1 peptide
LGSI

SEQ ID NO: 36 - protein, 4 aa, Artificial Sequence
  Modified Delta V1-1 peptide
LGSV

SEQ ID NO: 37 - protein, 11 aa, Artificial Sequence
  Modified Delta V1-2 peptide
ALSTDRGKTLV

SEQ ID NO: 38 - protein, 11 aa, Artificial Sequence
  Modified Delta V1-2 peptide
ALTSDRGKTLV

SEQ ID NO: 39 - protein, 11 aa, Artificial Sequence
  Modified Delta V1-2 peptide
ALTTDRGKSLV

SEQ ID NO: 40 - protein, 11 aa, Artificial Sequence
  Modified Delta V1-2 peptide
ALTTDRPKTLV

SEQ ID NO: 41 - protein, 11 aa, Artificial Sequence
  Modified Delta V1-2 peptide
ALTTDRGRTLV

SEQ ID NO: 42 - protein, 11 aa, Artificial Sequence
  Modified Delta V1-2 peptide
ALTTDKGKTLV

SEQ ID NO: 43 - protein, 11 aa, Artificial Sequence
  Modified Delta V1-2 peptide
ALTTDKGKTLV

SEQ ID NO: 44 - protein, 14 aa, Artificial Sequence
  Modified Delta V1-5 peptide
RAEFWLDLQPQAKV

SEQ ID NO: 45 - protein, 14 aa, Artificial Sequence
  Modified Delta V1-5 peptide
KADFWLDLQPQAKV

SEQ ID NO: 46 - protein, 14 aa, Artificial Sequence
  Modified Delta V1-5 peptide
KAEFWLELQPQAKV

SEQ ID NO: 47 - protein, 14 aa, Artificial Sequence
  Modified Delta V1-5 peptide
KAEFWLDLQPQARV

SEQ ID NO: 48 - protein, 14 aa, Artificial Sequence
  Modified Delta V1-5 peptide
KAEYWLDLQPQAKV

SEQ ID NO: 49 - protein, 14 aa, Artificial Sequence
  Modified Delta V1-5 peptide
KAEFWIDLQPQAKV

SEQ ID NO: 50 - protein, 14 aa, Artificial Sequence
  Modified Delta V1-5 peptide
KAEFWVDLQPQAKV

SEQ ID NO: 51 - protein, 14 aa, Artificial Sequence
  Modified Delta V1-5 peptide
KAEFWLDIQPQAKV

SEQ ID NO: 52 - protein, 14 aa, Artificial Sequence
  Modified Delta V1-5 peptide
KAEFWLDVQPQAKV

SEQ ID NO: 53 - protein, 14 aa, Artificial Sequence
  Modified Delta V1-5 peptide
KAEFWLDLNPQAKV

SEQ ID NO: 54 - protein, 14 aa, Artificial Sequence
  Modified Delta V1-5 peptide
KAEFWLDLQPNAKV

SEQ ID NO: 55 - protein, 14 aa, Artificial Sequence
  Modified Delta V1-5 peptide
KAEFWLDLQPQAKI

SEQ ID NO: 56 - protein, 14 aa, Artificial Sequence
  Modified Delta V1-5 peptide
KAEFWLDLQPQAKL

SEQ ID NO: 57 - protein, 14 aa, Artificial Sequence
  Modified Delta V1-5 peptide
KAEFWADLQPQAKV

SEQ ID NO: 58 - protein, 14 aa, Artificial Sequence
  Modified Delta V1-5 peptide
KAEFWLDAQPQAKV

SEQ ID NO: 59 - protein, 14 aa, Artificial Sequence
  Modified Delta V1-5 peptide
KAEFWLDLQPQAKA

SEQ ID NO: 60 - protein, 7 aa, Artificial Sequence
  Modified Delta V1-5 peptide
KAEFWLD

SEQ ID NO: 61 - protein, 8 aa, Artificial Sequence
  Modified Delta V1-5 peptide
DLQPQAKV

SEQ ID NO: 62 - protein, 8 aa, Artificial Sequence
  Modified Delta V1-5 peptide
EFWLDLQP

SEQ ID NO: 63 - protein, 7 aa, Artificial Sequence
  Modified Delta V1-5 peptide
LDLQPQA

SEQ ID NO: 64 - protein, 7 aa, Artificial Sequence
  Modified Delta V1-5 peptide
LQPQAKV

SEQ ID NO: 65 - protein, 7 aa, Artificial Sequence
  Modified Delta V1-5 peptide
AEFWLDL

SEQ ID NO: 66 - protein, 7 aa, Artificial Sequence
  Modified Delta V1-5 peptide
WLDLQPQ

SEQ ID NO: 67 - protein, 8 aa, Artificial Sequence
  Fragment of Delta V5 peptide
SPRPYSNF

SEQ ID NO: 68 - protein, 8 aa, Artificial Sequence
  Fragment of Delta V5 peptide
RPYSNFDQ

SEQ ID NO: 69 - protein, 8 aa, Artificial Sequence
  Fragment of Delta V5 peptide
SNFDQEFL

SEQ ID NO: 70 - protein, 8 aa, Artificial Sequence
  Fragment of Delta V5 peptide
DQEFLNEK

SEQ ID NO: 71 - protein, 8 aa, Artificial Sequence
  Fragment of Delta V5 peptide
FLNEKARL

SEQ ID NO: 72 - protein, 8 aa, Artificial Sequence
  Fragment of Delta V5 peptide
LIDSMDQS

SEQ ID NO: 73 - protein, 8 aa, Artificial Sequence
  Fragment of Delta V5 peptide
SMDQSAFA

SEQ ID NO: 74 - protein, 8 aa, Artificial Sequence
  Fragment of Delta V5 peptide
DQSAFAGF

SEQ ID NO: 75 - protein, 8 aa, Artificial Sequence
  Fragment of Delta V5 peptide
FVNPKFEH

SEQ ID NO: 76 - protein, 8 aa, Artificial Sequence
  Fragment of Delta V5 peptide
KFEHLLED

SEQ ID NO: 77 - protein, 8 aa, Artificial Sequence
  Fragment of Delta V5 peptide
NEKARLSY

SEQ ID NO: 78 - protein, 8 aa, Artificial Sequence
  Fragment of Delta V5 peptide
RLSYSDKN

SEQ ID NO: 79 - protein, 8 aa, Artificial Sequence
  Fragment of Delta V5 peptide
SYSDKNLI

SEQ ID NO: 80 - protein, 8 aa, Artificial Sequence
  Fragment of Delta V5 peptide
DKNLIDSM

SEQ ID NO: 81 - protein, 8 aa, Artificial Sequence
  Fragment of Delta V5 peptide
PFRPKVKS

SEQ ID NO: 82 - protein, 8 aa, Artificial Sequence
  Fragment of Delta V5 peptide
RPKVKSPR

SEQ ID NO: 83 - protein, 8 aa, Artificial Sequence
  Fragment of Delta V5 peptide
VKSPRPYS

SEQ ID NO: 84 - protein, 17 aa, Artificial Sequence
  Drosophila Antennapedia homeodomain-derived peptide
CRQIKIWFQNRRMKWKK

SEQ ID NO: 85 - protein, 11 aa, Artificial Sequence
  Transactivating Regulatory Protein (Tat)-derivedtransport peptide
YGRKKRRQRRR

SEQ ID NO: 86 - protein, 7 aa, Artificial Sequence
  Beta-II V5-3 peptide
CQEVIRN

SEQ ID NO: 87 - protein, 7 aa, Artificial Sequence
  Beta-II V5-3 peptide
CNEVIRN

SEQ ID NO: 88 - protein, 7 aa, Artificial Sequence
  Beta-II V5-3 peptide
CQDVIRN

SEQ ID NO: 89 - protein, 7 aa, Artificial Sequence
  Beta-II V5-3 peptide
CQEGIRN

SEQ ID NO: 90 - protein, 7 aa, Artificial Sequence
  Beta-II V5-3 peptide
CQEAIRN

SEQ ID NO: 91 - protein, 7 aa, Artificial Sequence
  Beta-II V5-3 peptide
CQELIRN

SEQ ID NO: 92 - protein, 7 aa, Artificial Sequence
  Beta-II V5-3 peptide
CQEIIRN

SEQ ID NO: 93 - protein, 7 aa, Artificial Sequence
  Beta-II V5-3 peptide
CQEVGRN

SEQ ID NO: 94 - protein, 7 aa, Artificial Sequence
  Beta-II V5-3 peptide
CQEVARN

SEQ ID NO: 95 - protein, 7 aa, Artificial Sequence
  Beta-II V5-3 peptide
CQEVVRN

SEQ ID NO: 96 - protein, 7 aa, Artificial Sequence
  Beta-II V5-3 peptide
CQELIRN

SEQ ID NO: 97 - protein, 7 aa, Artificial Sequence
  Beta-II V5-3 peptide
CQEVIKN

SEQ ID NO: 98 - protein, 7 aa, Artificial Sequence
  Beta-II V5-3 peptide
CQEVIHN

SEQ ID NO: 99 - protein, 7 aa, Artificial Sequence
  Beta-II V5-3 peptide
CQEVIRQ

SEQ ID NO: 100 - protein, 6 aa, Artificial Sequence
  Beta-II V5-3 peptide
QEVIRN

SEQ ID NO: 101 - protein, 7 aa, Artificial Sequence
  Beta-II V5-3 peptide
CNDVIRN

SEQ ID NO: 102 - protein, 8 aa, Artificial Sequence
  Beta-II V5-3 peptide
CNEVGIRN

SEQ ID NO: 103 - protein, 8 aa, Artificial Sequence
  Beta-II V5-3 peptide
CNEVAIRN

SEQ ID NO: 104 - protein, 8 aa, Artificial Sequence
  Beta-II V5-3 peptide
CNEVLIRN

SEQ ID NO: 105 - protein, 8 aa, Artificial Sequence
  Beta-II V5-3 peptide
CNEVVIRN

SEQ ID NO: 106 - protein, 8 aa, Artificial Sequence
  Beta-II V5-3 peptide
CNEVIIRN

SEQ ID NO: 107 - protein, 7 aa, Artificial Sequence
  Beta-II V5-3 peptide
CNEVIKN

SEQ ID NO: 108 - protein, 7 aa, Artificial Sequence
  Beta-II V5-3 peptide
CNEVIHN

SEQ ID NO: 109 - protein, 7 aa, Artificial Sequence
  Beta-II V5-3 peptide
CNEVIRQ

SEQ ID NO: 110 - protein, 8 aa, Artificial Sequence
  Beta-II V5-3 peptide
CQDVGIRN

SEQ ID NO: 111 - protein, 8 aa, Artificial Sequence
  Beta-II V5-3 peptide
CQDVAIRN

SEQ ID NO: 112 - protein, 8 aa, Artificial Sequence
  Beta-II V5-3 peptide
CQDVLIRN

SEQ ID NO: 113 - protein, 8 aa, Artificial Sequence
  Beta-II V5-3 peptide
CQDVVIRN

SEQ ID NO: 114 - protein, 8 aa, Artificial Sequence
  Beta-II V5-3 peptide
CQDVIIRN

SEQ ID NO: 115 - protein, 7 aa, Artificial Sequence
  Beta-II V5-3 peptide
CQDVIKN

SEQ ID NO: 116 - protein, 7 aa, Artificial Sequence
  Beta-II V5-3 peptide
CQDVIHN

SEQ ID NO: 117 - protein, 7 aa, Artificial Sequence
  Beta-II V5-3 peptide
CQDVIRQ

SEQ ID NO: 118 - protein, 7 aa, Artificial Sequence
  Beta-II V5-3 peptide
CQEGGRN

SEQ ID NO: 119 - protein, 7 aa, Artificial Sequence
  Beta-II V5-3 peptide
CQEGARN

SEQ ID NO: 120 - protein, 7 aa, Artificial Sequence
  Beta-II V5-3 peptide
CQEGVRN

SEQ ID NO: 121 - protein, 7 aa, Artificial Sequence
  Beta-II V5-3 peptide
CQEGLRN

SEQ ID NO: 122 - protein, 7 aa, Artificial Sequence
  Beta-II V5-3 peptide
CQEAGRN

SEQ ID NO: 123 - protein, 7 aa, Artificial Sequence
  Beta-II V5-3 peptide
CQEAARN

SEQ ID NO: 124 - protein, 7 aa, Artificial Sequence
  Beta-II V5-3 peptide
CQEAVRN

SEQ ID NO: 125 - protein, 7 aa, Artificial Sequence
  Beta-II V5-3 peptide
CQEALRN

SEQ ID NO: 126 - protein, 7 aa, Artificial Sequence
  Beta-II V5-3 peptide
CQEIGRN

SEQ ID NO: 127 - protein, 7 aa, Artificial Sequence
  Beta-II V5-3 peptide
CQEIARN

SEQ ID NO: 128 - protein, 7 aa, Artificial Sequence
  Beta-II V5-3 peptide
CQEILRN

SEQ ID NO: 129 - protein, 7 aa, Artificial Sequence
  Beta-II V5-3 peptide
CQELGRN

SEQ ID NO: 130 - protein, 7 aa, Artificial Sequence
  Beta-II V5-3 peptide
CQELARN

SEQ ID NO: 131 - protein, 7 aa, Artificial Sequence
  Beta-II V5-3 peptide
CQELVRN

SEQ ID NO: 132 - protein, 7 aa, Artificial Sequence
  Beta-II V5-3 peptide
CQELLRN

SEQ ID NO: 133 - protein, 7 aa, Artificial Sequence
  Beta-II V5-3 peptide
CQELGRN

SEQ ID NO: 134 - protein, 7 aa, Artificial Sequence
  Beta-II V5-3 peptide
CQELARN

SEQ ID NO: 135 - protein, 7 aa, Artificial Sequence
  Beta-II V5-3 peptide
CQELVRN

SEQ ID NO: 136 - protein, 7 aa, Artificial Sequence
  Beta-II V5-3 peptide
CQEVVKN

SEQ ID NO: 137 - protein, 7 aa, Artificial Sequence
  Beta-II V5-3 peptide
CQEVIKN

SEQ ID NO: 138 - protein, 7 aa, Artificial Sequence
  Beta-II V5-3 peptide
CQEVIKQ

SEQ ID NO: 139 - protein, 1009 aa, Homo sapiens
MSGVSEPLSRVKLGTLRRPEGPAEPMVVVPVDVEKEDVRILKVCFYSNSFNPGKNFKLVKCTVQTEIREIITSILLSGRIGPNIRLAECYGLRLKHMKSDEIHWLHPQMTVGEVQDKYECLHVEAEWRYDLQIRYLPEDFMESLKEDRTTLLYFYQQLRNDYMQRYASKVSEGMALQLGCLELRRFFKDMPHNALDKKSNFELLEKEVGLDLFFPKQMQENLKPKQFRKMIQQTFQQYASLREEECVMKFFNTLAGFANIDQETYRCELIQGWNITVDLVIGPKGIRQLTSQDAKPTCLAEFKQIRSIRCLPLEEGQAVLQLGIEGAPQALSIKTSSLAEAENMADLIDGYCRLQGEHQGSLIIHPRKDGEKRNSLPQIPMLNLEARRSHLSESCSIESDIYAEIPDETLRRPGGPQYGIAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAMEQERNARYRTPKILEPTAFQEPPPKPSRPKYRPPPQTNLLAPKLQFQVPEGLCASSPTLTSPMEYPSPVNSLHTPPLHRHNVFKRHSMREEDFIQPSSREEAQQLWEAEKVKMRQILDKQQKQMVEDYQWLRQEEKSLDPMVYMNDKSPLTPEKEVGYLEFTGPPQKPPRLGAQSIQPTANLDRTDDLVYLNVMELVRAVLELKNELCQLPPEGYVVVVKNVGLTLRKLIGSVDDLLPSLPSSSRTEIEGTQKLLNKDLAELINKMRLAQQNAVTSLSEECKRQMLTASHTLAVDAKNLLDAVDQAKVLANLAHPPAE

SEQ ID NO: 140 - protein, 1009 aa, Rattus norvegics
MSGVSEPLSRVKVGTLRPPEGPPEPMVVVPVDVEKEDVRILKVCFYSNSFNPGKNFKLVKCTVQTEIQEIITSILLSGRIGPNIQLAECYGLRLKHMKSDEIHWLHPQMTVGEVQDKYECLHVEAEWRYDLQIRYLPEDFMESLKEDRTTLLYFYQQLRNDYMQRYASKVSEGMALQLGCLELRRFFKDMPHNALDKKSNFELLEKEVGLDLFFPKQMQENLKPKQFRKMIQQTFQQYASLREEECVMKFFNTLAGFANIDQETYRCELIQGWNITVDLVIGPKGIRQLTSQDTKPTCLAEFKQIRSIRCLPLEETQAVLQLGIEGAPQSLSIKTSSLAEAENMADLIDGYCRLQGEHKGSLIIHAKKDGEKRNSLPQIPTLNLESRRSHLSESCSIESDIYAEIPDETLRRPGGPQYGVAREDVVLNRILGEGFFGEVYEGVYTNHKGEKINVAVKTCKKDCTLDNKEKFMSEAVIMKNLDHPHIVKLIGIIEEEPTWIVMELYPYGELGHYLERNKNSLKVPTLVLYALQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPELCPPVLYTLMTRCWDYDPSDRPRFTELVCSLSDIYQMERDIAIEQERNARYRPPKILEPTAFQEPPPKPSRPKYKHPPQTNLLAPKLQFQVPEGLCASSPTLTSPMEYPSPVNSLHTPPLHRHNVFKRHSMREEDFIRPSSREEAQQLWEAEKIKMRQVLDRQQKQMVEDSQWLRREERCLDPMVYMNDKSPLTPEKEAGYTEFTGPPQKPPRLGAQSIQPTANLDRTDDLVYHNVMTLVEAVLELKNKLSQLPPEEYVVVVKNVGLNLRKLIGSVDDLLPSLPASSRTEIEGTQKLLNKDLAELINKMRLAQQNAVTSLSEDCKRQMLTASHTLAVDAKNLLDAVDQAKVVANLAHPPAE

SEQ ID NO: 141 - protein, 1009 aa, Mus musculuc
MSGVSEPLSRVKVGTLRRPEGPPEPMVVVPVDVEKEDVRILKVCFYSNSFNPGKNFKLVKCTVQTEIQEIITSILLSGRIGPNIQLAECYGLRLKHMKSDEIHWLHPQMTVGEVQDKYECLHVEAEWRYDLQIRYLPEDFMESLKEDRTTLLYFYQQLRNDYMQRYASKVSEGMALQLGCLELRRFFKDMPHNALDKKSNFELLEKEVGLDLFFPKQMQENLKPKQFRKMIQQTFQQYASLREEECVMKFFNTLAGFANIDQETYRCELIQGWNITVDLVIGPKGIRQLTSQDTKPTCLAEFKQIRSIRCLPLEETQAVLQLGIEGAPQSLSIKTSSLAEAENMADLIDGYCRLQGEHKGSLIMHAKKDGEKRNSLPQIPTLNLEARRSHLSESCSIESDIYAEIPDETLRRPGGPQYGVAREEVVLNRILGEGFFGEVYEGVYTNHKGEKINVAVKTCKKDCTQDNKEKFMSEAVIMKNLDHPHIVKLIGIIEEEPTWIIMELYPYGELGHYLERNKNSLKVPTLVLYTLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPELCPPVLYTLMTRCWDYDPSDRPRFTELVCSLSDIYQMEKDIAIEQERNARYRPPKILEPTTFQEPPPKPSRPKYRPPPQTNLLAPKLQFQVPEGLCASSPTLTSPMEYPSPVNSLHTPPLHRHNVFKRHSMREEDFIRPSSREEAQQLWEAEKIKMKQVLERQQKQMVEDSQWLRREERCLDPMVYMNDKSPLTPEKEAGYTEFTGPPQKPPRLGAQSIQPTANLDRTDDLVYHNVMTLVEAVLELKNKLGQLPPEDYVVVVKNVGLNLRKLIGSVDDLLPSLPASSRTEIEGTQKLLNKDLAELINKMKLAQQNAVTSLSEDCKRQMLTASHTLAVDAKNLLDAVDQAKVVANLAHPPAE

SEQ ID NO: 142 - protein, 6 aa, Artificial sequence
  Betta-II V5-3 peptide
QEVIRN

SEQ ID NO: 143 - protein, 23 aa, Artificial Sequence
  Delta V1-1/TAT peptide
YGRKKRRQRRRCCSFNSYELGSL

SEQ ID NO: 144 - protein, 21 aa, Artificial Sequence
  Delta V1-7 TAT peptide
YGRKKRRQRRRCCMRAAEDPM

SEQ ID NO: 145 - protein, 19 aa, Artificial Sequence
  Betta V5-3/TAT peptide
YGRKKRRQRRRCCQEVIRN

SEQ ID NO: 146 - protein, 8 aa, Artificial Sequence
  Betta V5-3/TAT polypeptide
MRAAEDPM

SEQ ID NO: 147 - protein, 673 aa, Homo sapiens
MAPFLRISFNSYELGSLQAEDDASQPFCAVKMKEALTTDRGKTLVQKKPTMYPEWKSTFDAHIYEGRVIQIVLMRAAEDPMSEVTVGVSVLAERCKKNNGKAEFWLDLQPQAKVLMCVQYFLEDGDCKQSMRSEEEAMFPTMNRRGAIKQAKIHYIKNHEFIATFFGQPTFCSVCKEFVWGLNKQGYKCRQCNAAIHKKCIDKIIGRCTGTATNSRDTIFQKERFNIDMPHRFKVYNYMSPTFCDHCGSLLWGLVKQGLKCEDCGMNVHHKCREKVANLCGINQKLLAEALNQVTQKASRKPETPETVGIYQGFEKKTAVSGNDIPDNNGTYGKIWEGSNRCRLENFTFQKVLGKGSFGKVLLAELKGKERYFAIKYLKKDVVLIDDDVECTMVEKRVLALAWENPFLTHLICTFQTKDHLFFVMEFLNGGDLMFHIQDKGRFELYRATFYAAEIICGLQFLHGKGIIYRDLKLDNVMLDKDGHIKIADFGMCKENIFGENRASTFCGTPDYIAPEILQGLKYSFSVDWWSFGVLLYEMLIGQSPFHGDDEDELFESIRVDTPHYPRWITKESKDIMEKLFERDPAKRLGVTGNIRLHPFFKTINWNLLEKRKVEPPFKPKVKSPSDYSNFDPEFLNEKPQLSFSDKNLIDSMDQTAFKGFSFVNPKYEQFLE

SEQ ID NO: 148 - protein, 676 aa, Rattus norvegicus
MAPFLRIAFNSYELGSLQAEDEANQPFCAVKMKEALSTERGKTLVQKKPTMYPEWKSTFDAHIYEGRVIQIVLMRAAEEPVSEVTVGVSVLAERCKKNNGKAEFWLDLQPQAKVLMSVQYFLEDVDCKQSMRSEDEAKFPTMNRRGAIKQAKIHYIKNHEFIATFFGQPTFCSVCKDFVWGLNKQGYKCRQCNAAIHKKCIDKIIGRCTGTAANSRDTIFQKERFNIDMPHRFKVHNYMSPTFCDHCGSLLWGLVKQGLKCEDCGMNVHHKCREKVANLCGINQKLLAEALNQVTQRASRRSDSASSEPVGIYQGFEKKTGVAGEDMQDNSGTYGKIWEGSSKCNINNFIFHKVLGKGSFGKVLLGELKGRGEYFAIKALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGVTGNIKIHPFFKTINWTLLEKRRLEPPFRPKVKSPRDYSNFDQEFLNEKARLSYSDKNLIDSMDQSAFAGFSFVNPKFEHLLED

SEQ ID NO: 149 - protein, 7 aa, Homo sapiens
CQEIVRN

(19) United States
(12) Patent Application Publication
(10) Pub. No.: US 2009/0048174 A1
(43) Pub. Date: Feb. 19, 2009

(54) Methods for inhibiting angiogenesis and tumor growth by inhibition of beta or delta protein kinase C

(76) Inventors: Daria D. Mochly-Rosen, Melo Park, CA (US); Sylvia Jeewon Kim, San Jose, CA (US)

Correspondence Address: PERKINS COIE LLP, P.O. BOX 1208, SEATTLE, WA, 98111-1208, US

(21) Appl. No.: 11/999,806

(22) Filed: Dec. 7, 2007

Related U.S. Application Data
(60) Provisional application No. 60/873,762, filed on Dec. 8, 2006.
(60) Provisional application No. 60/875,227, filed on Dec. 15, 2006.

(51) Int. Cl. A61K 38/08; A61K 38/00; A61K 38/10; A61P 35/00

(52) U.S. Cl. 514/13; 514/2; 514/15; 514/17

(57) ABSTRACT

Treatment methods for inhibiting tumor growth and angiogenesis are described. The methods involve treatment with an inhibitor of delta protein kinase C (δPKC) or an inhibitor of beta-II protein kinase C (β_{II}PKC), in an amount effective to decrease the rate of growth of a solid tumor and/or to inhibit tumor angiogenesis.

### CROSS-REFERENCE TO RELATED APPLICATIONS

This application claims the benefit of U.S. Provisional Application Ser. Nos. 60/873,762, filed Dec. 8, 2006, and 60/875,227, filed Dec. 15, 2006, which are hereby incorporated by reference in their entirety.

### STATEMENT REGARDING GOVERNMENT INTEREST

This work was supported in part by National Cancer Institute, PHS Grant number CA09151. Accordingly the United States government may have certain rights in this invention.

### TECHNICAL FIELD

The subject matter described herein relates to treatment methods for inhibiting tumor growth and inhibiting angiogenesis. The methods involve administering an inhibitor of delta protein kinase C (δPKC) or an inhibitor of beta-II protein kinase C (β_{II}PKC), in an amount effective to decrease the rate of growth of a solid tumor and/or to inhibit tumor angiogenesis.

### BACKGROUND

Angiogenesis is the physiological process by which new blood vessels develop from pre-existing vessels. A wide variety of human diseases are characterized by unregulated blood vessel development, including ocular diseases such as macular degeneration and diabetic retinopathy, and tumor growth. The growth of solid tumors appears to require new blood vessel growth (i.e., angiogenesis) to support the continued expansion of the tumor beyond a minimal size. Blocking tumor neovascularization can significantly inhibit tumor growth (Varner, J. A. et al. (1995) Cell Adh. Commun. 3:367).

Tumor metastasis is the process by which malignant cells from a tumor spread throughout the body and develop into multiple secondary tumors (Lida et. al. (1996) Sem. Cancer Biol. 7:155-62). In order to spread to other parts of the body, tumor cells escape from the primary or original tumor, enter the blood stream or lymphatic system, and from there invade the tissue of other organs, where they may form new tumors. Escape from the primary tumor and invasion into other organs is a complex multi-step process. Metastasis involves changes in tumor cell adhesion and motility and the secretion of proteolytic enzymes, chemoattractants, and proteoglycans. Angiogenesis, or the formation of new blood vessels, is also a vital step in the metastatic process (Folkman, J. (1995) Nature Medicine 1:27-31).

Prostate cancer is the second leading cause of cancer-related deaths in the U.S., with over 234,960 new incidents occurring each year. Treatment involves androgen deprivation therapy to reduce the proliferation of androgen-dependent prostate cancer cells. While often effective for the first few years following diagnosis, tumors frequently become resistant to therapy (i.e., androgen-independent). In addition, androgen deprivation is associated with various side effects, including osteoporosis, hot flashes, loss of libido, erectile dysfunction, depression, and anemia.

Metastatic prostate cancer is usually resistant to treatment with current chemotherapeutic agents, which produce only a moderate improvement in patient survival rate associated at the expense of increased risk of neutropenia, neuropathy and edema. This chemoresistance may be due to indolent characteristic of prostate cancer. Agents that confer superior therapeutic effects on advanced prostate cancer and extend the window for treating the condition with therapeutic agents are greatly needed.

As mentioned several times, angiogenesis plays an important role in solid tumor growth, including prostate cancer tumor growth. Advanced and metastatic prostate cancer tumors require angiogenesis to permit them to grow beyond a small nodule. Immunohistochemical studies show an increase in microvessel density with prostate cancer progression. In general, angiogenesis and the expression of pro-angiogenic factors are associated with adverse outcomes in prostate cancer patients. In pre-clinical models, angiogenesis inhibitors have been shown to be effective against prostate cancer. Anti-angiogenic therapy is cytostatic, not cytotoxic like chemotherapy, and therefore betted suited for treating slow growing tumors like prostate cancer tumors. The development of new pharmacological treatments that target tumor cell proliferation and angiogenesis are greatly needed.

The protein kinase C (PKC) family of serine/theronine kinases has been repeatedly implicated in the mechanisms that regulate tumor cell growth, survival and tumor-induced angiogenesis. Over 20 years ago, based on activation of PKC by tumor promoting phorbol-esters, it was suggested that activation of PKC may be involved in carcinogenesis (Castagna, M. et al. (1982) J. Biol. Chem. 257:7847-51). PKC activation contributes to tumor progression of many human cancers. In particular, βPKC activation has been reported in diffuse large B-cell lymphomas (Hans, C. P. et al. (2005) Mod. Pathol. 18:1377-84), glioblastoma, colon cancer, and renal cancer (Graff, J. R. et al. (2005) Cancer Res. 65:7462-69 and Keyes, et al. (2004) Cancer Chemother Pharmacology 53:133-140. βPKC has also been repeatedly implicated in tumor-induced angiogenesis and tumorigenesis (Yoshiji, H. et al. (1999) Cancer Res. 59:4413-18; Graff, J. R. et al. (2005) Cancer Res. 65:7462-69; and Green, L. J. et al. (2006) Clin. Cancer Research 12:3408-15).

The PKC family includes ten different isozymes. In prostate tumors, isozymes α, β, δ, ε, ζ, λ/ι, and μ have been reported (Cornford, P. et al. (1999) Am. J. Pathol. 154:137-144 and Koren, R. et al. (2004) Oncol. Rep. 11:321-6). However, whether the alterations in the levels of PKC isozymes occur in the tumor cells or in the surrounding microvasculature is unknown, as are the reasons for the changes in isozyme levels as the tumors progress.

It would be desirable to have a method of inhibiting angiogenesis and tumor growth utilizing compounds that selectively inhibit particular PKC isozymes in tumor cells and/or its supporting vasculature.

### BRIEF SUMMARY

The following aspects of the invention and embodiments thereof described and illustrated below are intended to be exemplary and illustrative, not limiting in scope.

In one aspect, the invention provides a treatment method comprising administering an inhibitor of delta protein kinase C (δPKC) or an inhibitor of beta-II protein kinase C (β_{II}PKC) in an amount effective to decrease the rate of growth of a solid tumor. In another aspect, the invention provides a treatment method, comprising administering an inhibitor of delta protein kinase C or an inhibitor of beta-II protein kinase C (β_{II}PKC) in an amount effective to inhibit tumor angiogenesis.

In one preferred embodiment of the treatment methods, the inhibitor of δPKC is a peptide. In some embodiments, the peptide is selected from the first variable region of δPKC. In particular embodiments, the peptide is a peptide having between about 5 and 15 contiguous residues from the first variable region of δPKC. In a related embodiment, the peptide has at least about 50% sequence identity with a conserved set of between about 5 and 15 contiguous residues from the first variable region of δPKC. In particular embodiments, the peptide has at least about 80% sequence identity with SFNSYELGSL (SEQ ID NO:1).

In another preferred embodiment of the treatment methods, the inhibitor of β_{II}PKC is a peptide. In some embodiments, the peptide is selected from the fifth variable region of β_{II}PKC. In particular embodiments the peptide is a peptide having between about 5 and 15 contiguous residues from the fifth variable region of β_{II}PKC. In related embodiments, the peptide has at least about 50% sequence identity with a conserved set of between about 5 and 15 contiguous residues from the fifth variable region of β_{II}PKC. In a particular embodiment, the peptide has at least about 80% sequence identity with QEVIRN (SEQ ID NO: 142).

In some embodiments of the invention, the peptide inhibitor of δPKC or β_{II}PKC is modified to include a terminal Cys residue. In one particular embodiment, peptide is modified to include an N-terminal Cys residue. In some embodiments, the peptide is modified to include a carrier molecule. In particular embodiments of the invention, the carrier molecule is selected from a Drosophila Antennapedia homeodomain-derived sequence (CRQIKIWFQNRRMKWKK, SEQ ID NO: 84), a Transactivating Regulatory Protein (Tat)-derived transport polypeptide from the Human Immunodeficiency Virus, Type 1 (YGRKKRRQRRR, SEQ ID NO: 85), or a polyarginine.

In some embodiments of the invention, the solid tumor is a tumor of the prostate. In many embodiments, angiogenesis is associated with a tumor or a tumor cell in the prostate. In particular embodiments, tumor angiogenesis is associated with a metastasized tumor cell.

In addition to the exemplary aspects and embodiments described above, further aspects and embodiments will become apparent by reference to the drawings and by study of the following descriptions.

### BRIEF DESCRIPTION OF THE DRAWINGS

FIG. 1 is a graph showing the levels of β_{II}PKC in the particulate fraction over total level of prostate cancer cells (PC3, solid bar) and of immortalized normal prostate cells (PZ, open bar).

FIGS. 2A-2D show immunoblot analysis of cytosolic (FIGS. 2A-2B) and particulate (FIGS. 2C and 2D) fractions of prostate cancer cells using an anti-β_{I}PKC antibody (FIGS. 2A and 2C) or an anti-β_{II}PKC antibody (FIGS. 2B and 2D).

FIG. 2E is a bar graph showing the levels of β_{II}PKC (solid bar) and β_{I}PKC (open bar), determined based on the immunoblot analysis shown in FIGS. 2A-2D. Isozyme levels refer to the fraction of isozyme that is in particulate (identified as Triton-soluble (TS) over Total) with respect to β_{I}PKC or β_{II}PKC.

FIGS. 3A and 3B show immunoblot analysis of the cytosolic (FIG. 3A) and the particulate fraction (FIG. 3B) of prostate cancer cells grown in vivo for 3, 4, 6, and 8 weeks. The blots were probed with anti-β_{I}PKC antibody.

FIG. 3C is a bar graph showing the levels of β_{II}PKC in prostate cancer cells grown in vivo for 3, 4, 6, and 8 weeks, determined based on the immunoblot analysis shown in FIGS. 3A and 3B. Isozyme levels refer to the fraction of isozyme that is in particulate, identified as particulate/(cytosolic+particulate).

FIGS. 4A-4F show immunoblot analysis of the cytosolic fractions (FIGS. 4A, 4C, and 4E) and of the particulate fractions (FIGS. 4B, 4D, and 4F) of prostate cancer tissues. The blots were probed with an anti-αPKC antibody (FIGS. 4A and 4B), an anti-εPKC antibody (FIGS. 4C and 4D), or an anti-zetaPKC antibody (FIGS. 4E and 4F).

FIGS. 5A-5C are bar graphs showing the levels of αPKC (FIGS. 5A), εPKC (FIGS. 5B), and zetaPKC (FIGS. 5C) in prostate cancer cells grown in vivo for 4, 6, and 8 weeks. The levels were determined from the immunoblot analysis shown in FIGS. 4A-4F and expressed as the fraction in particulate (identified as particulate/total).

FIG. 6 is a graph showing weekly tumor volume (in mm³) following the injection of prostate cancer cells in mice during treatment with a control saline solution (open circles, upper line) or with β_{II}PKC peptide inhibitor β_{II}V5-3 administered from an implanted pump at a dose of 3 mM for 2 weeks and 30 mM for the following 3 weeks.

FIGS. 7A-7D show immunoblot analysis of the cytosolic (FIGS. 7A and 7B) and particulate (FIGS. 7C and 7D) fractions of prostate cancer cells harvested from mice following treatment for 3 weeks with a control saline solution (FIGS. 7A and 7C) or with β_{II}PKC peptide inhibitor (FIGS. 7B and 7D). The blots were probed with anti-β_{II}PKC antibody;

FIG. 7E is a bar graph showing the levels of β_{II}PKC in the prostate cancer cells obtained from the animals treated as described in FIGS. 7A-7D. Isozyme levels refer to the fraction in particulate, identified as particulate/(soluble+particulate).

FIGS. 8A-8D show immunoblot analysis of the cytosolic (FIGS. 8A and 8B) and of the particulate (FIGS. 8C and 8D) fractions of liver cells harvested from mice after treatment for 5 weeks with saline (FIGS. 8A and 8C) or β_{II}PKC peptide inhibitor (FIGS. 8B and 8D) and probed with an anti-β_{II}PKC antibody.

FIG. 8E is a bar graph showing the level of β_{II}PKC in liver cells harvested from animals treated as described in FIGS. 8A-8D. The levels were determined from the blots shown in FIGS. 8A-8D and reported as the ratio of β_{II}PKC in the particulate fraction of the liver cells to the total amount of the isozyme in the cytosol and particulate fractions, for the saline-treated control animals and the peptide inhibitor-treated animals.

FIGS. 9A-9D show immunoblot analysis of the cytosolic (FIGS. 9A and 9B) and particulate (FIGS. 9C and 9D) fractions of liver cells harvested from mice after treatment for 5 weeks with saline (FIGS. 9A and 9C) or β_{II}PKC peptide inhibitor (FIGS. 9B and 9D). The blots were probed with an anti-εPKC antibody.

FIG. 9E is a bar graph showing the level of εPKC in liver cells from animals treated as described in FIGS. 9A-9D. The levels of particulate isozyme were determined from the blots in FIGS. 9A-9D (identified as isozyme level in the particulate fractions over cytosol and particulate, for the saline-treated control animals and the peptide inhibitor-treated animals).

FIGS. 10A-10D show immunoblot analysis of cytosolic (FIGS. 10A and 10B) and particulate (FIGS. 10C and 10D) fractions of prostate cancer cells harvested from mice after treatment for 5 weeks with saline (FIGS. 10A and 10C) or with a β_{II}PKC peptide inhibitor (FIGS. 10B and 10D). The blots were probed with anti-β_{I}PKC antibody.

FIG. 10E is a bar graph showing the levels of β_{I}PKC in prostate cancer cells from animals treated as described in FIGS. 10A and 10D. The levels of the particulate fractions of β_{I}PKC are shown in the graph (identified as TS/total).

FIG. 11A is a graph showing the growth curve of tumor volume (in mm3) at various times during growth in the absence of treatment.

FIG. 11B is a graph showing the rate of tumor endothelial cell proliferation and of tumor cell proliferation (i.e., fractional turnover per day (k/day)) during the course of normal growth in nude mice in the absence of treatment.

FIG. 12 is a graph showing the tumor volume (in mm³) at various times during the continuous treatment of animals bearing a prostate cancer tumor with saline (diamonds) or a β_{II}PKC peptide inhibitor at a dose of 30 mM peptide at rate of administration was 0.5 μl/hr.

FIGS. 13A-13B are bar graphs showing the rate of tumor endothelial cell (TEC) proliferation (FIG. 13A) and of tumor cell (TC) proliferation (FIG. 13B), expressed as fractional turnover per day (k/day), in prostate cancer tumor cells harvested from mice following 3-weeks of continuous treatment with saline (open bars) or with a β_{II}PKC peptide inhibitor (solid bars).

FIGS. 14A-14B are bar graphs showing the concentration of vascular endothelial growth factor (VEGF, in pg/ml) in prostate cancer tumor cells harvested from mice following three-week (FIG. 14A) and six-week (FIG. 14B) continuous treatments with saline (open bars) or with a β_{II}PKC peptide inhibitor (solid bars).

FIG. 15 is a graph showing the level of δPKC in the particulate fraction of prostate tumor cells (solid bar) and immortalized normal prostate cells (open bar).

FIGS. 16A-16D show immunoblot analysis of the cytosolic (FIGS. 16A and 16B) and particulate (FIGS. 16C and 16D) fractions of prostate cancer cells harvested from mice following 3-8 weeks of normal tumor growth with no treatment. The blots were probed with an anti-δPKC antibody (FIGS. 16A and 16B) or with an anti-GAPDH antibody (FIGS. 16C and 16D).

FIG. 16E is a bar graph showing the levels of δPKC in prostate cancer cells harvested from animals treated as described in FIGS. 16A and 16D. The levels of the particulate fractions of δPKC are shown in the graph (identified as TS/total).

FIG. 17 is a graph showing tumor volume (in mm³) as a function of time at various times during the continuous treatment of animals bearing a prostate cancer tumor with a δPKC V1-1 peptide inhibitor (squares, lower line), δPKC V1-7 peptide activator (small half squares, upper line), or with a TAT carrier peptide (small squares, thin line).

FIGS. 18A-18D show immunoblot analysis of the cytosolic (FIGS. 18A and 18B) and particulate (FIGS. 18C and 18D) fractions of prostate cancer cells harvested from mice after treatment for 5 weeks with saline (FIGS. 18A and 18C) or with δPKC peptide activator (FIGS. 18B and 18D). The blot was probed with an anti-δPKC antibody.

FIG. 18E is a bar graph showing the levels of δPKC in prostate cancer cells from animals treated as described in FIGS. 18A and 18D. The levels of the particulate fractions of β_{I}PKC are shown in the graph (identified as particulate fraction/cytosolic+particulate (total)).

FIGS. 19A-19D show immunoblot analysis of the cytosolic (FIGS. 19A and 19B) and particulate (FIGS. 19C and 19D) fractions of prostate cancer cells harvested from mice after treatment for three 5 with saline (FIGS. 19A and 19C) or with δPKC peptide activator (FIGS. 19B and 19D). The blot was probed with an anti-εPKC antibody.

FIG. 19E is a bar graph showing the levels of εPKC in prostate cancer cells harvested from animals treated as described in FIGS. 19A-19D. The levels of the particulate fractions of β_{I}PKC are shown in the graph (identified as pellet/pellet+soluble).

FIG. 20 is a graph showing tumor volume (in mm³) at various times during the continuous treatment of animals bearing a prostate cancer tumor with a δPKC V1-7 peptide activator (upper line), with a TAT carrier peptide (circles, middle line), or with saline (circles, lower line).

FIG. 21 is a bar graph quantifying CD31 staining (tumor vessels) and

FIG. 22 is a graph showing the proliferation rate of tumor cells in animals bearing prostate cancer tumors and treated for five weeks with a δPKC peptide activator (solid bars) or with saline (open bars).

FIGS. 23A and 23B are bar graphs showing the concentration of vascular endothelial growth factor (VEGF, in pg/ml), in prostate cancer tumor cells in mice following 5-weeks of continuous treatment with saline (open bars) or with a δPKC peptide activator (solid bars). The levels of VEGF were measured after three weeks (FIG. 23A) and six weeks (FIG. 23B).

FIGS. 24A-24D show immunoblot analysis of extracts from tumor tissue obtained from saline-treated (FIGS. 24A and 24C) or δPKC peptide activator-treated (FIGS. 24B and 24D) animals. The blots were probed with antibodies specific for HIF-1a and GAPDH.

FIGS. 24A-24D show immunoblot analysis of the lysates of tumor cells extracted from tumor tissues with saline (open bar) or with δPKC peptide activator (solid bar). The levels were determined from the blots in FIGS. 24A and 24D.

FIG. 24E is a bar graph showing the levels of HIF-1 (normalized for GAPDH) in tumor tissue from animals treated with saline (open bar) or with δPKC peptide activator (solid bar). The levels were determined from the blots in FIGS. 24A and 24D.

FIGS. 25A and 25B are bar graphs showing the rate of proliferation of tumor endothelial cells (FIG. 25A) and of tumor cell proliferation (FIG. 25B), expressed as fractional turnover per day (k/day), in prostate cancer tumor cells in mice following 3-weeks of continuous treatment with saline (open bars) or with a δPKC peptide activator (solid bars).

FIG. 25C is a bar graph showing the tumor weight in animals treated with saline (open bars) or a δPKC peptide activator (solid bars). Tumor weight is in grams (Y-axis).

FIG. 26 is a graph showing the percent of TUNEL-positive cells in prostate cancer tumor cells in mice after treatment continuously for 5 weeks with saline (light circles) or with a δPKC peptide activator (squares).

FIGS. 27A-27C are graphs showing the relationship between apoptosis and tumor volume in tumor-bearing mice treated with saline (FIG. 27A) or with a δPKC peptide activator (FIG. 27B). FIG. 27C shows the combined data from both saline-treated and δPKC peptide activator-treated mice.

### DETAILED DESCRIPTION

### I. Definitions

Unless otherwise indicated, all terms should be given their ordinary meaning as known in the art (see, e.g., F. M. et al., John Wiley and Sons, Inc., Media Pa.) for definitions and terms of art. Abbreviations for amino acid residues are the standard 3-letter and/or 1-letter codes used in the art to refer to one of the 20 common L-amino acids.

A “conserved set” of amino acids refers to a contiguous sequence of amino acids that is identical or closely homologous (e.g., having only conservative amino acid substitutions) between members of a group of proteins. A conserved set may be anywhere from two to over 50 amino acid residues in length. Typically, a conserved set is between two and ten contiguous residues in length.

“Conservative amino acid substitutions” are substitutions that do not result in a significant change in the activity or tertiary structure of a selected polypeptide or protein. Such substitutions typically involve replacing a selected amino acid residue with a different residue having similar physico-chemical properties. For example, substitution of Glu for Asp is considered a conservative substitution since both are similarly-sized negatively-charged amino acids. Groupings of amino acids by physico-chemical properties are known to those of skill in the art.

“Domain” and “region” are used interchangeably herein and refer to a contiguous sequence of amino acids within a PKC isozyme, typically characterized by being either conserved or variable.

“Peptide” and “polypeptide” are used interchangeably herein and refer to a compound made up of a chain of amino acid residues linked by peptide bonds. Unless otherwise indicated, the sequence for peptides is given in the order from the “N” (or amino) termiums to the “C” (or carboxyl) terminus.

Two amino acid sequences or two nucleotide sequences are considered “homologous” (as this term is preferably used in this specification) if they have an alignment score of >5 (in standard deviation units) using the program ALIGN with the mutation gap matrix and a gap penalty of 6 or greater (Dayhoff, M. O., in ATLAS OF PROTEIN SEQUENCE AND STRUCTURE (1972) Vol. 5, National Biomedical Research Foundation, pp. 101-110, and Supplement 2 to this volume, pp. 1-10.) The two sequences (or parts thereof are more preferably homologous if their amino acids are greater than or equal to 50%, more preferably 70%, still more preferably 80%, identical when optimally aligned using the ALIGN program mentioned above.

A peptide or peptide fragment is “derived from” a parent peptide or polypeptide if it has an amino acid sequence that is homologous to the amino acid sequence of, or is a conserved fragment from, the parent peptide or polypeptide.

“Modulate” intends a lessening, an increase, or some other measurable change in PKC activation, tumor cell proliferation, morbidity, mortality, etc.

“Management,” for example in the context of treating pain, intends both a lessening of pain and/or induction of analgesia.

The term “treatment” or “treating” means any treatment of disease in a mammal, including: (a) preventing or protecting against the disease, that is, causing the clinical symptoms not to develop; (b) inhibiting the disease, that is, arresting or suppressing the development of clinical symptoms; and/or (c) relieving the disease, that is, causing the regression of clinical symptoms. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

The term “effective amount” means a dosage sufficient to provide treatment for the disorder or disease state being treated. This will vary depending on the patient, the disease and the treatment being effected.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The following abbreviations are defined for clarity:

| Abbreviation | Meaning |
|---|---|
| l | liter |
| ml | milliliter |
| μl | microliter |
| M | molar |
| mM | millimolar |
| μM | micromolar |
| nM | nanomolar |
| pM | picomolar |
| g | gram |
| mg | milligram |
| μg | microgram |
| a.a. | amino acid |
| min | minute(s) |
| sec or s | second(s) |
| wks | weeks |
| α | alpha |
| β | beta |
| δ | delta (“d” in some |
| | figure legends) |
| ε | epsilon |
| sal | usually saline |

### II. Methods of Treatment

A. Treatment of Animals with a Beta_{II}-PKC Inhibitor

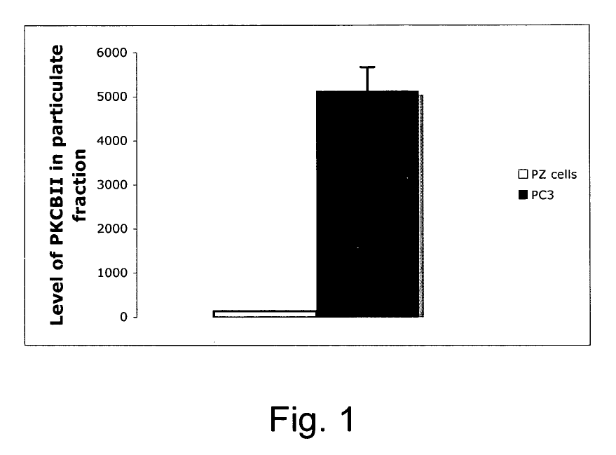

FIG. 1 is a graph showing the levels of β_{II}PKC in immortalized normal prostate epithelial cells (PZ, open bar) and androgen-independent prostate cancer cells (PC3, solid bar). The levels of β_{II}PKC are many times greater in prostate cancer cells than in normal immortalized prostate epithelial cells.

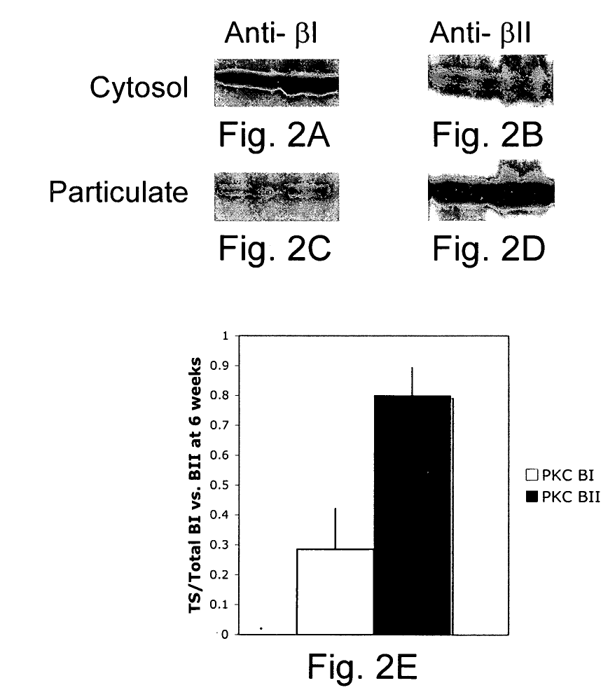

FIGS. 2A-2D show the results of immunoblot (i.e. western blot) analysis using cytosolic cell fractions (FIGS. 2A and 2B) and insoluble (particulate) cell fractions (FIGS. 2C and 2D) from PC3 prostate cancer cells, along with an antibody specific for β_{I}PKC (FIGS. 2A and 2C) or β_{II}PKC (FIGS. 2B and 2D). The results show higher levels of cytosolic β_{I}PKC compared to those of particulate β_{I}PKC, and higher levels of particulate β_{II}PKC than those of cytosolic β_{II}PKC in PC3 cells. FIG. 2E is a bar graph comparing the levels of particulate β_{I}PKC (open bar) and β_{II}PKC (solid bar) relative to the total levels (cytosolic and particulate) of each protein kinase, based on the data shown in FIGS. 2A-2D. The results from FIGS. 2A-2E show that increased levels of particulate β_{II}PKC and decreased levels of particulate β_{I}PKC are associated with prostate tumors.

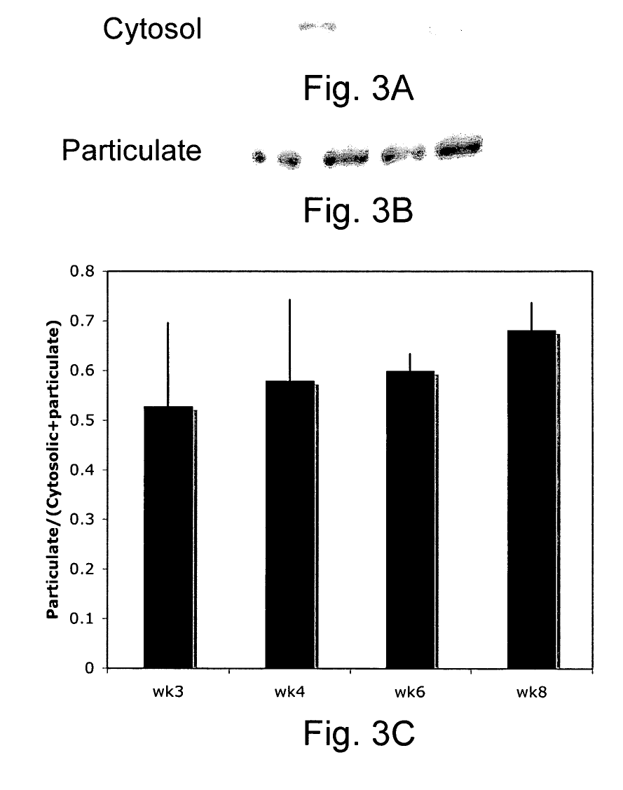

FIGS. 3A and 3B show the results of immunoblot analysis using cytosolic cell fractions (FIG. 3A) and particulate cell fractions (FIG. 3B) obtained from PC3 prostate cancer cells grown in culture for 4, 6, or 8 weeks. The blots were probed with an antibody specific for β_{I}PKC. FIG. 3C is a bar graph showing the levels of particulate β_{II}PKC relative to the total levels level of β_{II}PKC, based on the data shown in FIGS. 3A and 3B. These results indicate that the levels of particulate β_{II}PKC increase over time in growing prostate tumor cells. These cell culture results suggest that the progression of prostate cancer in animals is characterized by escalating levels of particulate β_{II}PKC.

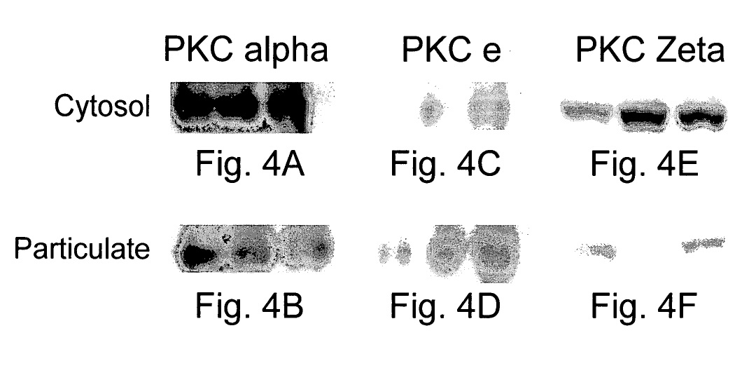

FIGS. 4A-4F show the results of immunoblot analysis using cytosolic cell fractions (FIGS. 4A, 4C, and 4E) and particulate cell fractions (FIGS. 4B, 4D, and 4F) obtained from PC3 prostate cancer cells following 6-weeks of growth in vivo. The blots were probed with an antibody specific for αPKC (FIGS. 4A and 4B), εPKC (FIGS. 4C and 4D), or zetaPKC (FIGS. 4E and 4F). The results show higher levels of cytosolic αPKC compared to particulate αPKC, similar levels of cytosolic εPKC compared to particulate εPKC, and higher levels of cytosolic zetaPKC compared to particulate zetaPKC, in PC3 prostate tumor cells.

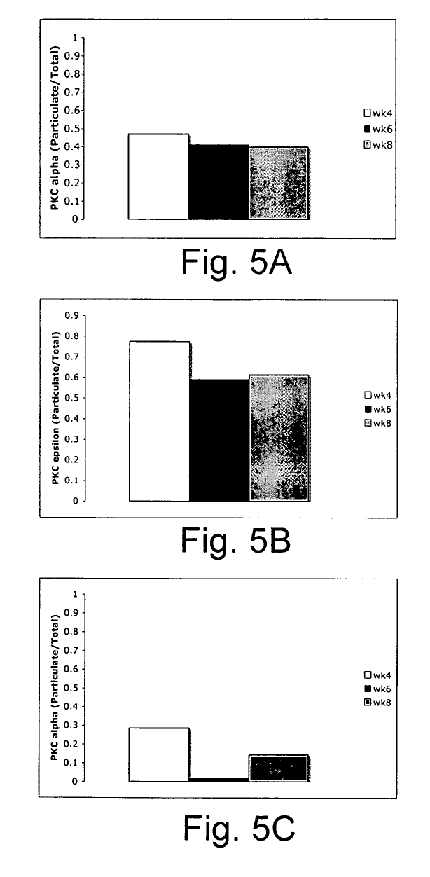

FIGS. 5A-5C are bar graphs showing the relative levels of particulate αPKC (FIG. 5A), εPKC (FIG. 5B), and zetaPKC (FIG. 5C) compared to the total levels for each protein kinase, in prostate cancer cells grown in culture for 4 (open bars), 6 (dark bars), and 8 (gray/medium bars) weeks. The results show that the levels of these three protein kinase C isozymes do not increase over time as the prostate cancer cells (PC3) are grown in vivo, contrary to the levels of β_{II}PKC.

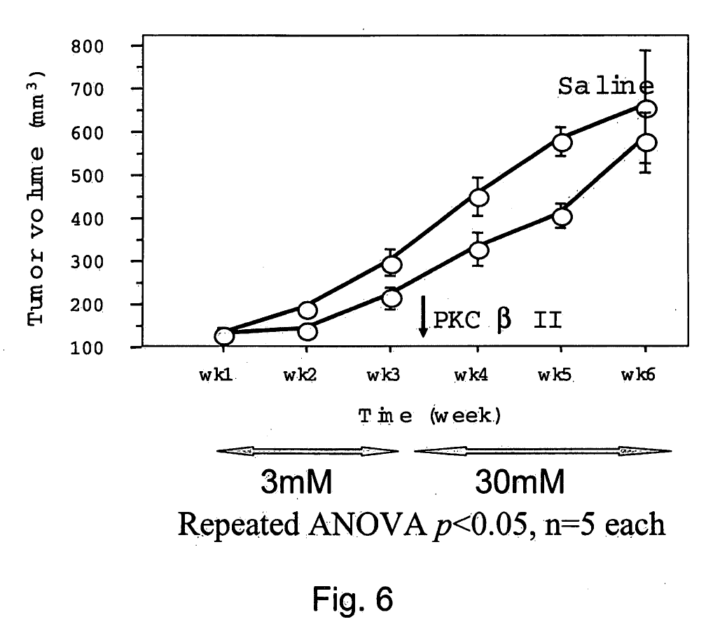

FIG. 6 is a graph of tumor volume (in mm³) as a function of time (in weeks) following injection of PC3 prostate cancer cells into mice (i.e., a xenograft), which were treated with a saline solution as a control (open circles, upper line) or with β_{II}PKC peptide inhibitor β_{II}V5-3, having the amino acid sequence CQEVIRN (SEQ ID NO:86; Stebbins, E. G. and Mochly-Rosen, D. (2001) J. Biol. Chem. 276:29644-50), which was administered from an implanted pump at a dose of 3 mM for two weeks and 30 mM for an additional three weeks. The results show that the continuous administration of a β_{II}PKC peptide inhibitor reduces the growth rate of tumors in animals.

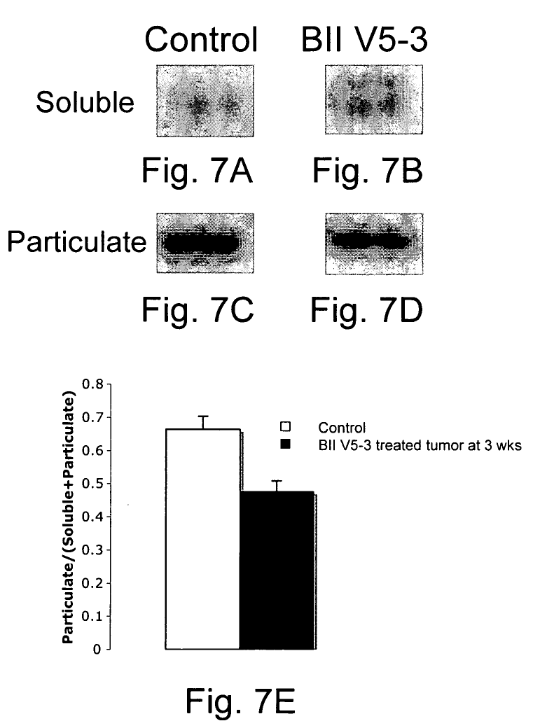

FIGS. 7A-7D show the results of immunoblot analysis using cytosolic (soluble) cell fractions (FIGS. 7A and 7B) and particulate cell fractions (FIGS. 7C and 7D) obtained from PC3 prostate cancer isolated from the animals described in FIG. 6 at 3 weeks following treatment with β_{II}PKC peptide inhibitor β_{II}V5-3 or saline solution (as a control). The blots were probed with an antibody specific for β_{II}PKC. FIG. 7E is a bar graph showing the levels of particulate β_{II}PKC relative to the total levels level of β_{II}PKC, in β_{II}PKC peptide inhibitor-treated and untreated control animals, based on the data shown in FIGS. 7A-7D. The levels of particulate β_{II}PKC in treated animals were only 72% of those in untreated animals (p<0.05). The results show that levels of particulate β_{II}PKC decrease following treatment with the β_{II}PKC peptide inhibitor.

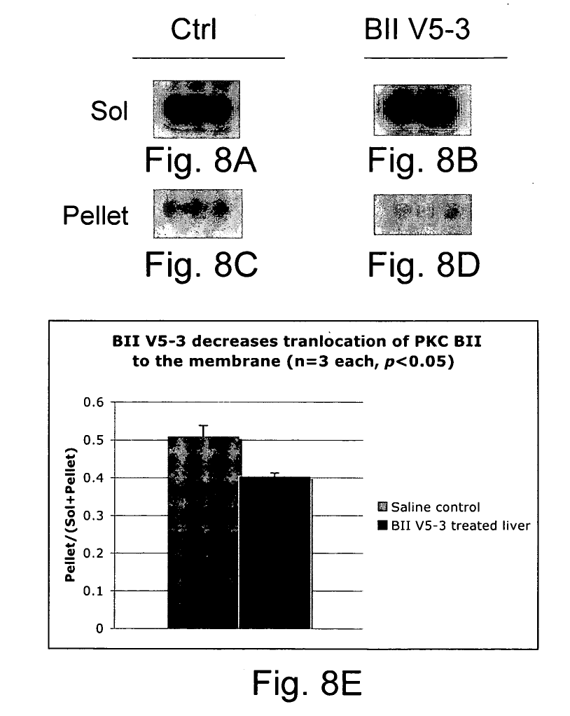

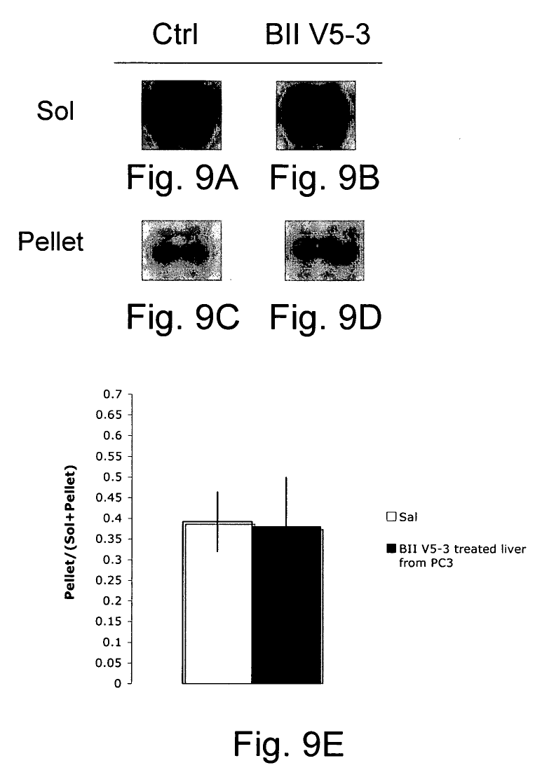

FIGS. 8A-8D show the results of immunoblot analysis using cytosolic (soluble) cell fractions (FIGS. 8A and 8B) and particulate (pellet) cell fractions (FIGS. 8C and 8D) obtained from liver cells obtained from 5-week β_{II}PKC peptide inhibitor-treated animals (FIGS. 8B and 8D) and untreated animals (FIGS. 8A and 8C) shown in FIG. 6 after 5 weeks of treatment. The blots were probed with an antibody specific for β_{II}PKC. FIG. 8E is a bar graph showing the levels of particulate β_{II}PKC relative to the total levels level of β_{II}PKC in these animals. Untreated animals are represented by open bars. Treated animals are represented by solid bars. The results show that levels of particulate β_{II}PKC decrease as a result of β_{II}PKC peptide inhibitor-treatment.

FIGS. 9A-9D show the results of immunoblot analysis using cytosolic (soluble) cell fractions (FIGS. 9A and 9B) and particulate fractions (FIGS. 9C and 9D) of liver cells harvested from the animals shown in FIG. 6 following treatment for 5 weeks with the β_{II}PKC peptide inhibitor (FIGS. 9B and 9D) or a saline control (FIGS. 9A and 9C). The blots were probed with an antibody specific for εPKC. FIG. 9E is a bar graph showing the levels of particulate εPKC relative to the total levels level of εPKC in these animals. The results show that the levels of particulate εPKC in the liver do not substantially change following treatment with β_{II}PKC peptide inhibitor β_{II}V5-3.

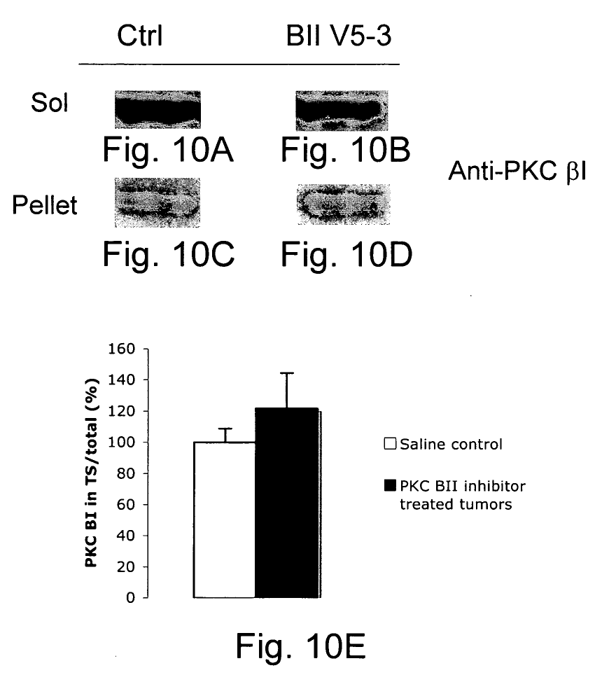

FIGS. 10A-10D show the results of immunoblot analysis using cytosolic (soluble) cell fractions (FIGS. 10A and 10B) and particulate fractions (FIGS. 10C and 10D) of prostate cancer cells harvested from mice following treatment for 5 weeks with a saline control (FIGS. 10A, 10C) or with β_{II}PKC peptide inhibitor β_{II}V5-3 (FIGS. 10B and 10D). The blots were probed with antibody specific for β_{I}PKC. FIG. 10E is a bar graph showing the levels of particulate β_{I}PKC relative to the total levels level of β_{I}PKC in these animals. Untreated animals are represented by open bars. Treated animals are represented by solid bars. The results show that levels of particulate β_{I}PKC increases slightly following β_{II}PKC peptide inhibitor-treatment.

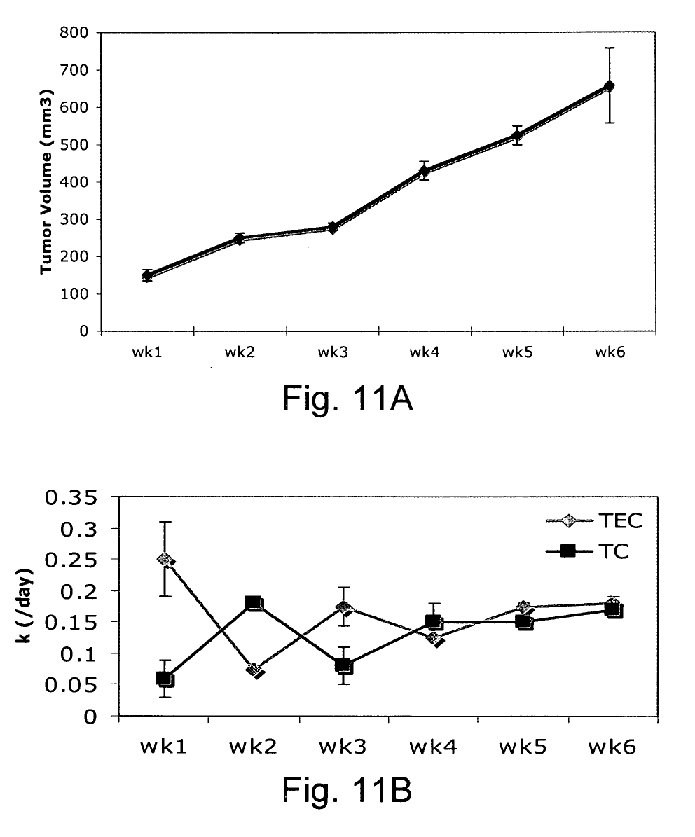

FIG. 11A is a graph showing tumor volume (in mm³) as a function of time (in weeks) at various times in the absence of treatment. FIG. 11B is a graph showing the rate of tumor endothelial cell (TEC, closed diamonds) and tumor cell (TC, closed squares) proliferation in these animals (fractional turnover per day (k/day)). The results show a roughly weekly cycle of alternating TEC and TC proliferation, which is most pronounced up to about four weeks following treatment and less pronounced after about 4 weeks of treatment.

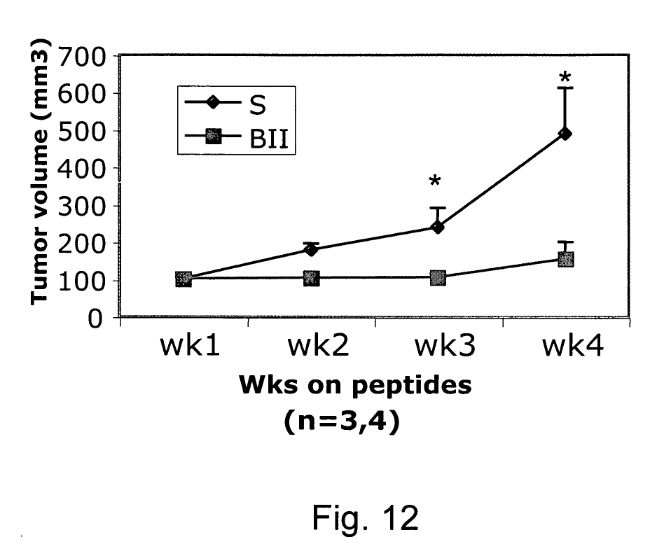

FIG. 12 is a graph showing tumor volume (in mm³) in the weeks following treatment with a higher dose of β_{II}PKC peptide inhibitor β_{II}V5-3 (i.e., 30 mM at rate of administration was 0.5 μl/hr). Animals treated with saline solution as a control are indicated by closed diamonds, while animals treated with the β_{II}PKC peptide inhibitor are indicated by closed squares. The results show that increasing the dosage of the β_{II}PKC peptide inhibitor further increases the therapeutic effect, in terms of reducing the volume of the prostate cancer tumor (e.g., compared to the result shown in FIG. 6).

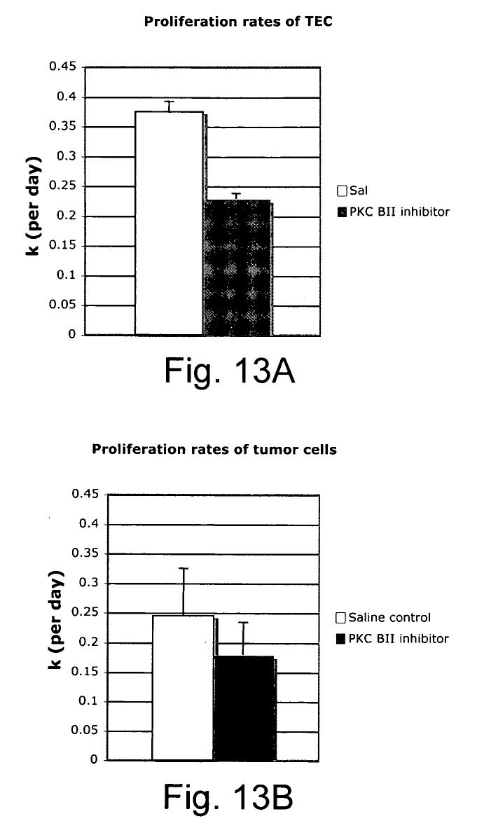

FIGS. 13A and 13B are bar graphs showing the rates of tumor endothelial cell (TEC) proliferation (FIG. 13A) and tumor cell (TC) proliferation (FIG. 13B), expressed as fractional turnover per day (k/day), in mixed tumor cells obtained from animals after three weeks of continuous treatment with saline solution as a control (open bars) or with a β_{II}PKC peptide inhibitor (solid bars). The results show a decrease in both endothelial cell and tumor cell proliferation as a results of β_{II}PKC peptide inhibitor treatment.

Tumor cells (mixed cell populations) obtained from control and β_{II}PKC peptide inhibitor β_{II}V5-3-treated animals following 5-weeks of treatment were subjected to histological analysis to determine the effect of the β_{II}PKC peptide inhibitor on apoptosis (data not shown). CD31 is a tumor endothelial marker used to identify tumor cells in a sample. CD31 (PECAM-1) has been implicated in angiogenesis, apoptosis, cell migration, modulation of integrin-mediated cell adhesion, transendothelial migration, negative regulation of immune cell signaling, autoimmunity, macrophage phagocytosis, IgE-mediated anaphylaxis, and thrombosis. Terminal deoxynucleotidyl transferase-mediated dUTP nick end labeling (i.e., TUNEL labeling) is used to detect the formation of DNA fragments, which are characteristic of cells undergoing apoptosis. Hoechst staining is used to measure chromatin condensation, which is also characteristic of apoptotic cells. The cleavage of caspase-3 is yet another indicator of apoptosis.

Tumor cell samples obtained from control animals showed increased staining for CD31 compared to equivalent tumor cell samples obtained from β_{II}PKC peptide inhibitor-treated animals, suggesting reduced vascularization in tumors obtained from β_{II}PKC peptide inhibitor-treated animals. In contrast, the same tumor cell samples obtained from β_{II}PKC peptide inhibitor-treated animals showed increased TUNEL labeling in endothelium, Hoechst staining, and caspase 3 cleavage compared to tumor cell samples obtained from control animals. These results indicate that tumor endothelial cells growing in animals treated with a β_{II}PKC peptide inhibitor show increased levels of apoptosis compared to tumors from untreated animals.

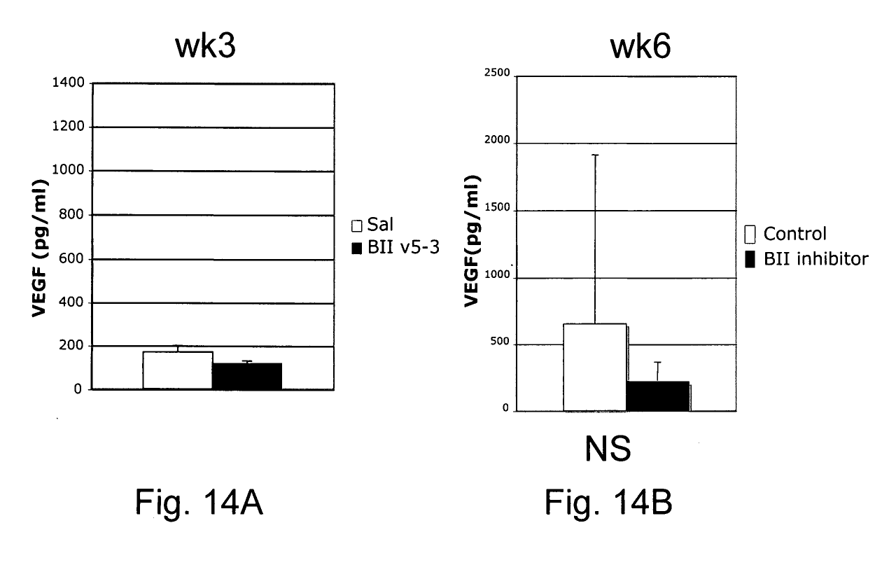

FIGS. 14A 14B are bar graphs showing the concentration of vascular endothelial growth factor (VEGF; in pg/ml) in prostate cancer tumor cells obtained from animals treated continuously for three weeks (FIG. 14A) or six weeks (FIG. 14B) with a control saline solution (open bars) or with a β_{II}PKC peptide inhibitor (solid bars). VEGF is associated with vascularization. The levels of VEGF were lower in β_{II}PKC peptide inhibitor-treated animals at both three and six weeks following treatment, with the difference being more pronounced at six weeks. These results show that treatment with the β_{II}PKC peptide inhibitor reduced VEGF expression in tumor cells, thereby reducing vascularization of the tumor.

B. Treatment of Animals with a Delta-PKC Inhibitor

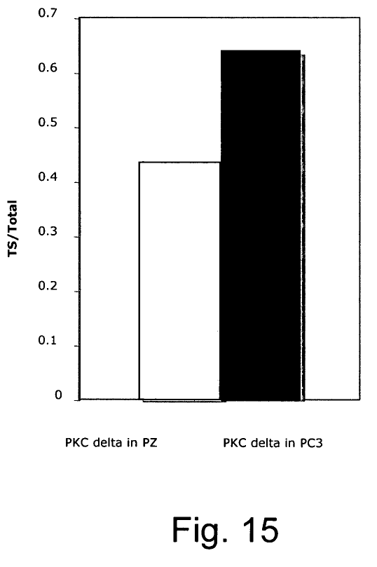

FIG. 15 is a graph showing the relative levels of particulate δPKC compared to total δPKC in immortalized normal prostate epithelial cells (PZ, open bar) and androgen-independent prostate cancer cells (PC3, solid bar). The results demonstrate that the levels of particulate δPKC are greater in prostate cancer cells than in normal immortalized prostate epithelial cells.

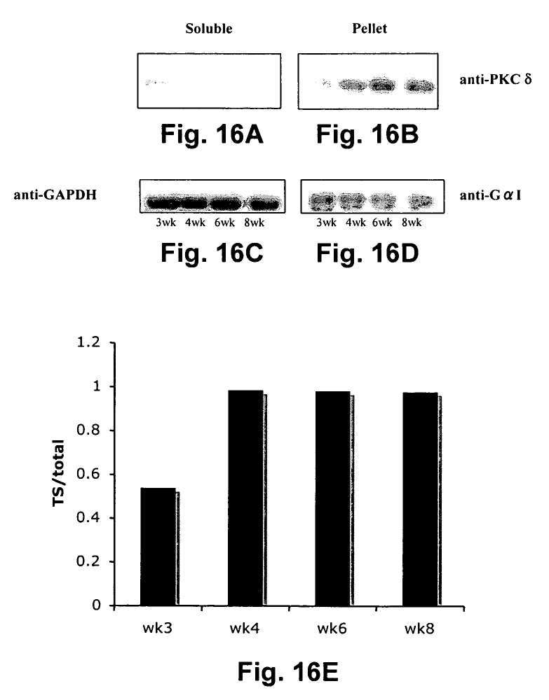

FIGS. 16A-16D show the results of immunoblot analysis using cytosolic (soluble) cell fractions (FIGS. 16A and 16C) and particulate fractions (FIGS. 16B and 16D) obtained from PC3 tumor xenografts grown in vivo for 3, 4, 6, or 8 weeks. The blots were probed with an antibody specific for δPKC (FIGS. 16A and 16B) or an antibody specific for GAPDH as a control (FIGS. 16C and 16D). FIG. 16E is a graph showing the relative levels of particulate δPKC compared to total δPKC, based on the data from FIGS. 16A-16D The results indicate that the levels of particulate δPKC initially increase when prostate tumor cells are grown in vivo, then level-off after about four weeks.

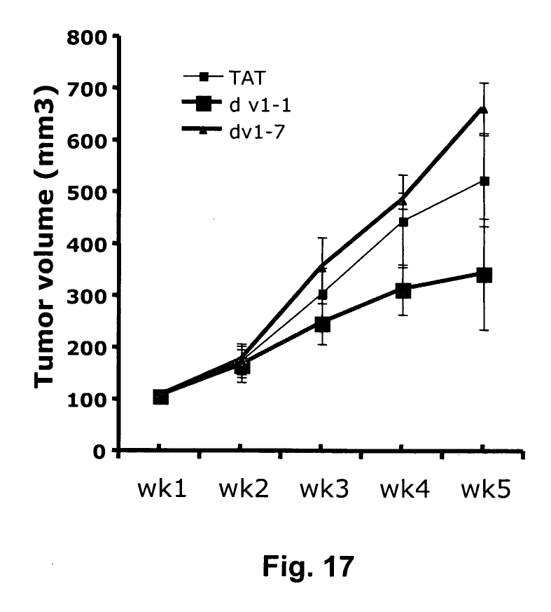

FIG. 17 is a graph showing tumor volume (in mm³) over the course of three weeks of treatment with an inhibitor of δPKC (δV1-1, large squares and heavy line) or an activator of δPKC (dV1-7, triangles). Each peptide was conjugated to TAT to facilitate uptake by cells. Control cells received TAT protein without a δPKC peptide (small squares). The δPKC activator caused an increase in tumor volume compared to control cells, while the δPKC inhibitor caused a decrease in tumor volume. These results show that a δPKC inhibitor reduces prostate tumor size in animals.

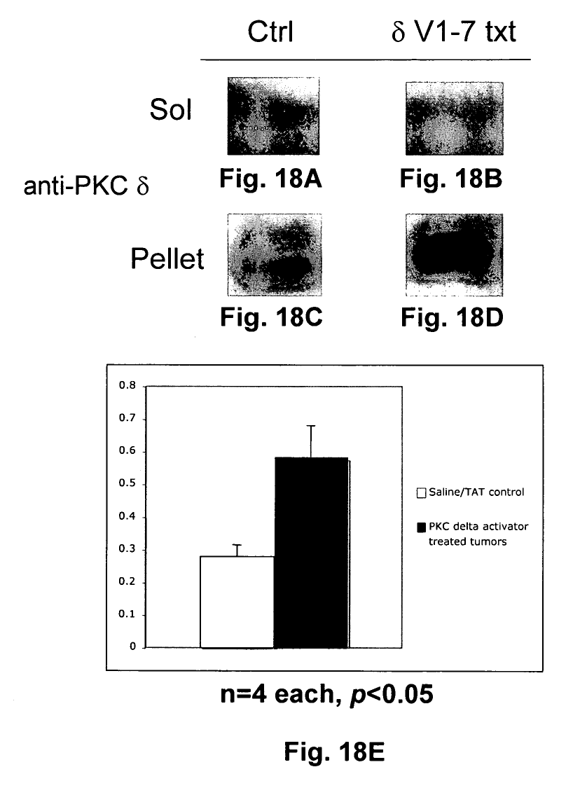

FIGS. 18A-18D show the results of immunoblot analysis using cytosolic (soluble) cell fractions (FIGS. 18A and 18B) and particulate fractions (FIGS. 18C and 18D) obtained from tumor cells isolated from the control (FIGS. 18A and 18C) or δPKC activator δV1-7-treated (FIGS. 18B and 18D) animals of FIG. 17 treated for 5 weeks. The blots were probed with an antibody specific for δPKC. The results show an increase in the levels of particulate δPKC (as a percentage of the total, Y-axis) following treatment with the δPKC activator. FIG. 18E is a graph showing the relative levels of particulate δPKC compared to total δPKC, based on the data from FIGS. 18A-18D. The levels of particulate δPKC are approximately doubled following treatment with the δPKC activator.

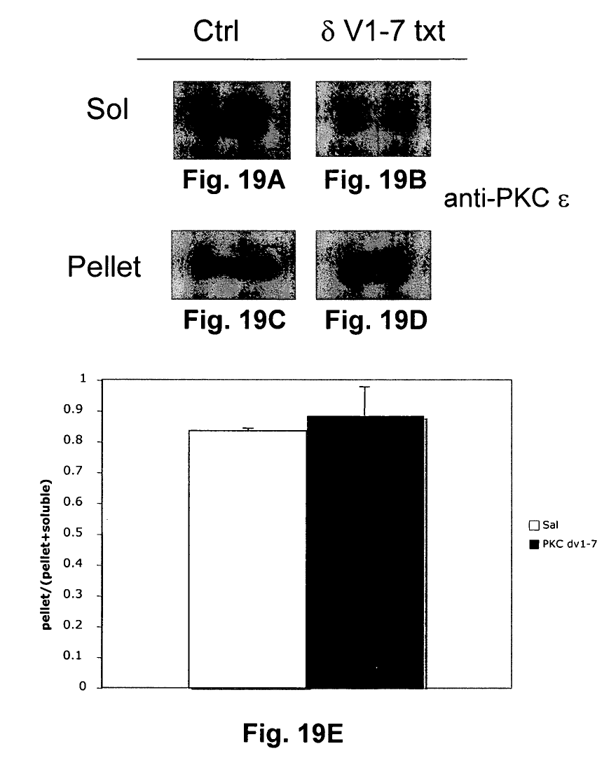

FIGS. 19A-19D show the results of immunoblot analysis using cytosolic (soluble) cell fractions (FIGS. 19A and 19B) and particulate fractions (FIGS. 19C and 19D) obtained from tumor cells isolated from the control (FIGS. 18A and 18C) or δPKC activator δV1-7-treated (FIGS. 18B and 18D) animals. The blots were probed with an antibody specific for εPKC. FIG. 19E is a graph showing the relative levels of particulate εPKC compared to total εPKC, based on the data from FIGS. 19A-19D. The results show that the levels of εPKC do not substantially change following treatment with the δPKC activator, indicating that the activator is specific for δPKC.

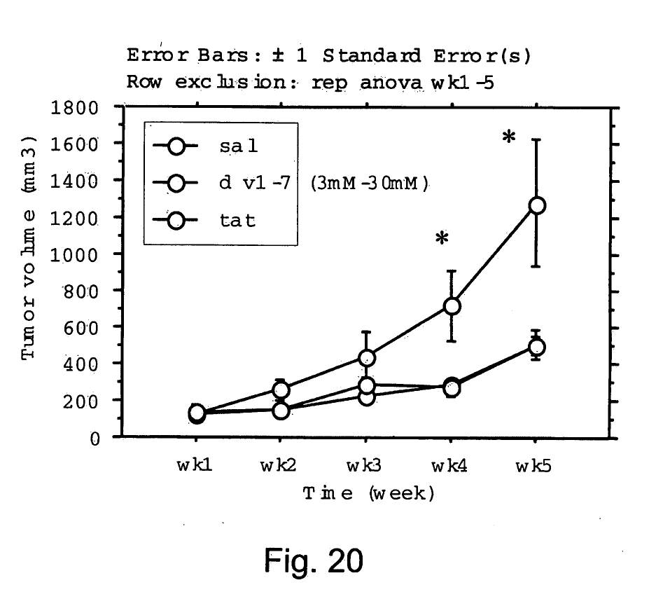

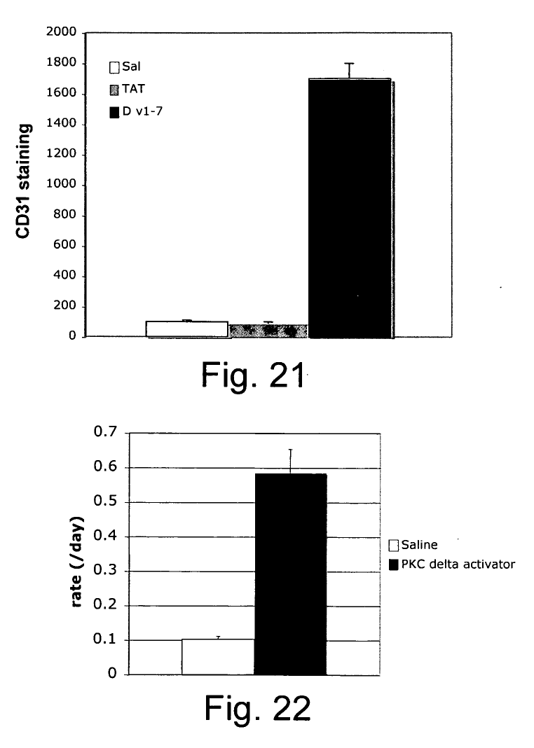

FIG. 20 is a graph showing tumor volume (in mm³) during the continuous treatment of animals with a control saline solution (dark open circles), Tat without a peptide inhibitor or activator (light grey circles), or the Tat-conjugated δPKC V1-7 peptide activator (medium grey circles). Treatment with the Tat-conjugated δPKC V1-7 peptide activator significantly increased tumor volume compared to the two groups of control animals (p=0.004). FIGS. 21 and 22 show further characterization of the tumor cell samples isolated from the control-treated animals and δPKC activator-treated animals from FIG. 20, following 5 weeks of treatment (i.e., at the end-stage of the experiment). FIG. 21 shows the results of CD31 staining of animals treated with a control saline solution (open bar), Tat without a peptide inhibitor or activator (grey bar), or the Tat-conjugated δPKC V1-7 (dark bar). Treatment with the δPKC activator cause a several-fold increase in tumor staining with CD31, suggesting increased vascularization in the δPKC activator treated tumors. FIG. 22 shows the rate of tumor cell proliferation in saline solution (open bar) or Tat-conjugated δPKC V1-7 (dark bar)-treated animals. δPKC peptide activator-treated animals show a substantial increase in tumor cell growth rate compared to the control animals.

Tumor tissue obtained from animals treated with a control saline solution or the δPKC activator peptide were stained with an antibody specific for Ki67 to detect proliferating cells in all phases of the cell cycle (i.e., G1, S-, G2-, and M-phase), but not in resting cells (G0-phase). The tumors obtained from activator-treated animals showed increased Ki67 staining, indicating the presence of more proliferating cells.

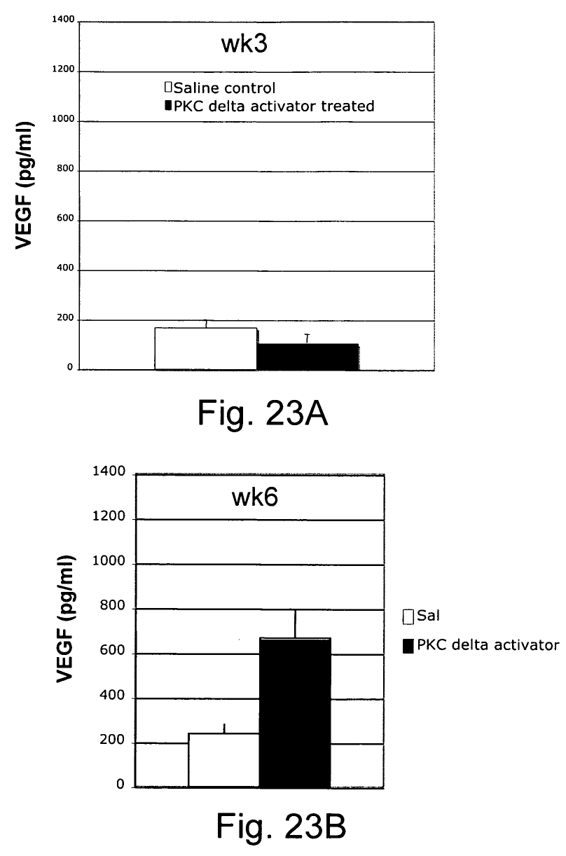

FIGS. 23A and 23B are bar graphs showing the concentration of vascular endothelial growth factor (VEGF, in pg/ml) in prostate cancer tumor cells in mice following three weeks continuous treatment with a control saline solution (open bars) or a δPKC peptide activator (solid bars). The levels of VEGF measured after three week of treatment and five weeks of treatment are shown in FIGS. 23A and 23B, respectively). The results show that angiogenesis is not increased after 3 weeks.

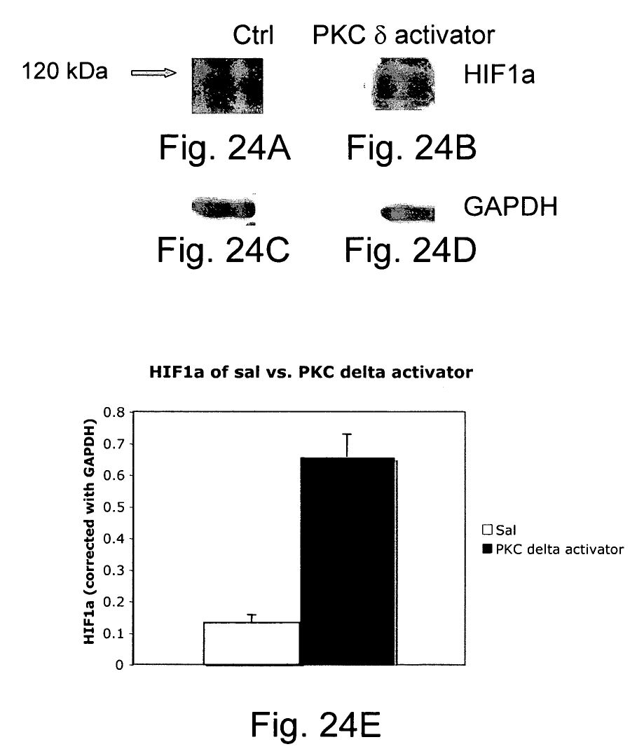

FIGS. 24A-24D show the results of immunoblot analysis total cell homogenate obtained from tumor cells from saline control (FIGS. 24 A and 24C) and δPKC activator (FIGS. 24 B and 24D) treated animals (5 weeks). The blots were probed with an antibody specific for hypoxia-inducible factors (HIF-1a, FIGS. 24A and 24B) or an antibody specific for GAPDH as a control (FIGS. 24C and 24D). FIG. 24E is a graph showing the relative levels of HIF-1a (normalized for GADPH) from FIGS. 24A-24D. The results show that treatment with the δPKC peptide activator causes a several-fold increase in the levels of HIF-1a (closed bar), compared to control-treated animals (open bar) (p<0.05).

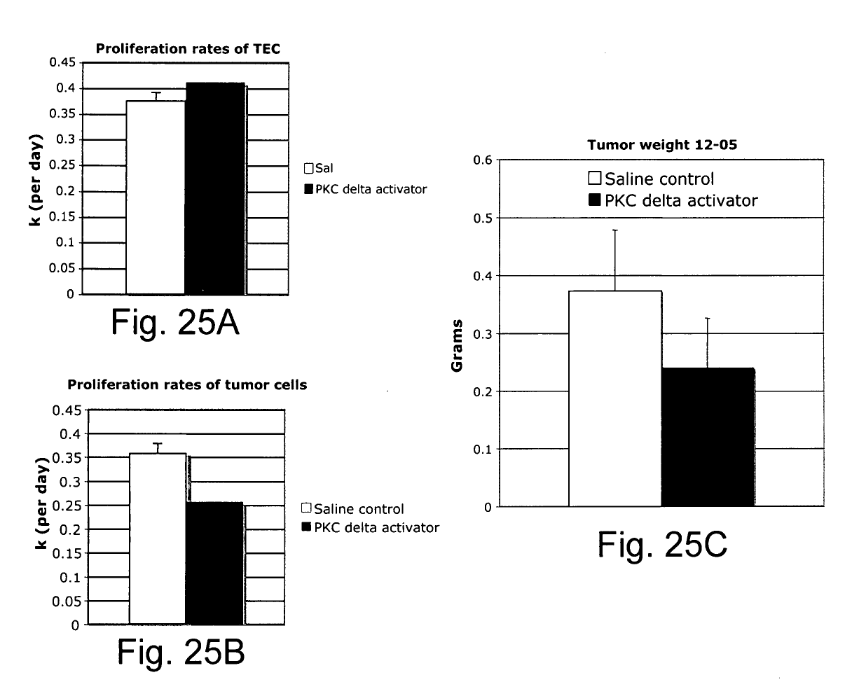

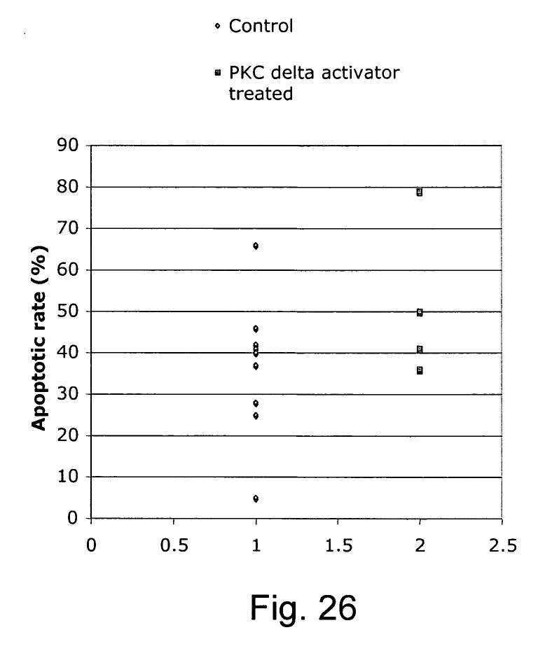

FIGS. 25A-25B are bar graphs showing the rate of proliferation (k/day) of tumor endothelial cells (TEC, FIG. 25A) and tumor cells (FIG. 25B), in prostate tumor cells obtained from animals following 3-weeks of treatment with a control saline solution (open bars) or with a δPKC peptide activator (solid bars). While the rate of proliferation (fractional turnover per day (k/day)) of tumor endothelial cells was similar in control and δPKC peptide activator-treated animals (FIG. 25A), the rate of proliferation of tumor cells appeared to decrease in δPKC peptide activator-treated animals (FIG. 25B). As shown in FIG. 25C, tumor mass (in grams) also decreased following δPKC peptide activator-treatment. These results suggested that the more rapid disease progression in δPKC peptide activator-treated animals is not apparent at early stage due to a suppression of net tumor cell proliferation by other mechanism.

To further investigate the mechanism by which the δPKC peptide activator affect tumor progression in animals, TUNEL labeling was performed on mixed tumor cell population obtained from saline control-treated (small circles) and δPKC peptide activator-treated (squares) animals (FIG. 26). The results were reported as the percentage of cells stained by TUNEL labeling. Treatment with the δPKC peptide activator increased TUNEL labeling only slightly, after 5-weeks treatment.

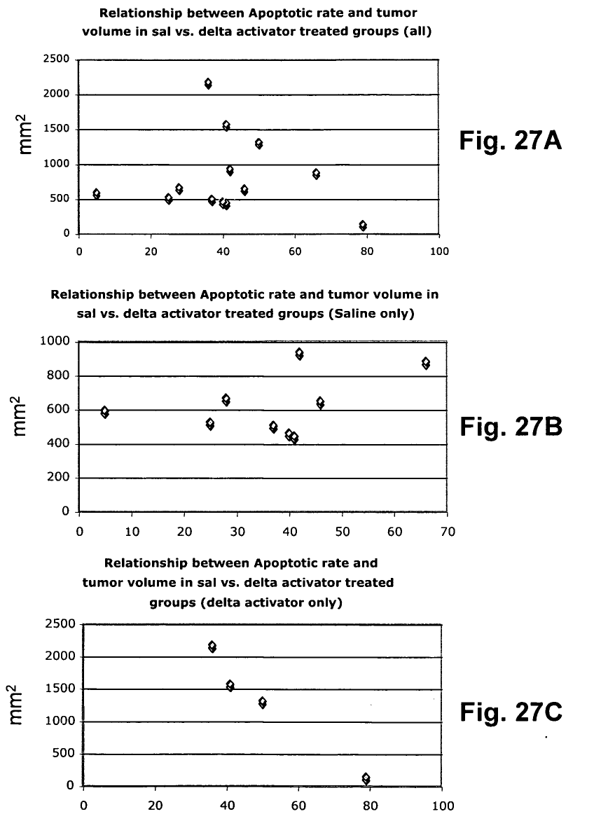

Further analysis of the data suggested that tumor cells obtained from δPKC peptide activator-treated animals were more resistant to apoptosis than cells from control-treated animals. FIGS. 27A-27C show tumor volume (mm²) as a function of the percent of TUNEL-positive cells (as in FIG. 26) for saline control treated animals (FIG. 27A), for δPKC peptide activator-treated animals (FIG. 27B), or for all animals (FIG. 27C). As shown in FIG. 27B, δPKC peptide activator-treated animals tended to have larger tumor volumes for a given percent of TUNEL-positive cells compared to control animals. These results suggest that δPKC peptide activator treatment causes tumor cells to be more resistant to apoptosis, thereby increasing overall tumor size and disease progression, which results in increased net proliferation rate of the tumor cells. This was not evident in early stage of tumor growth after 3-week treatment.

C. Summary of Results Using Beta_{II} and Delta PKC Inhibitors

The results show that increased β_{II}PKC protein levels, and increased relative levels of particulate β_{II}PKC, are found in prostate tumor cells (e.g., PC3 cells) but not immortalized normal prostate epithelial cells (PZ cells). Prostate tumor cells grown in vivo produce an increasing translocation of β_{II}PKC to particulate fraction. Treatment with a β_{II}PKC peptide inhibitor reduces the size of tumors, reduces the levels of VEGF expressed by tumor cells, and reduces angiogenesis in tumor tissue. Treatment with a β_{II}PKC peptide inhibitor also increases the level of apoptosis in tumors.

Increased levels of particulate δPKC are also associated with prostate tumor cells (PC3) compared to immortalized normal prostate cells (PZ). δPKC inhibitors and activators decrease or increase, respectively, overall tumor volume in animals. δPKC activation promotes angiogenesis by upregulating HIF-1a and VEGF. δPKC activation also causes prostate tumor cells to become more resistant to apoptosis.

These observations suggest that β_{II}PKC and δPKC are good drug targets and indicate that inhibitors of β_{II}PKC and δPKC can be used to reduce tumor size (i.e., treat tumor) in an animal.

D. Examples of PKC Inhibitors for Use with the Invention

A wide variety of inhibitors of β_{II}PKC and δPKC may be utilized to treat tumors in animals. As used herein, inhibitors of β_{II}PKC or δPKC are compounds that inhibit at least one biological activity or function of β_{II}PKC or δPKC. For example, inhibitors suitable for use with the present invention may inhibit the enzymatic activity of β_{II}PKC or δPKC (e.g., by preventing activation, binding to and/or phosphorylation of a protein substrate, inhibit the binding to the receptor for activated kinase (RACK), and or modulating the subcellular translocation of β_{II}PKC or δPKC.

In certain embodiments of the invention, a protein inhibitor of β_{II}PKC or δPKC may be utilized. The protein inhibitor may be in the form of a peptide. Proteins, polypeptides, and peptides (used without distinction with respect to inhibitors) are known in the art, and generally refer to compounds comprising amino acid residues linked by peptide bonds. Unless otherwise stated, the individual sequence of the peptide is given in the order from the amino terminus to the carboxyl terminus. Polypeptide/peptide inhibitors of β_{II}PKC δPKC may be obtained by methods known to the skilled artisan. For example, the peptide inhibitor may be chemically synthesized using various solid phase synthetic technologies known to the art and as described, for example, in Williams, Paul Lloyd, et al. Chemical Approaches to the Synthesis of Peptides and Proteins, CRC Press, Boca Raton, Fla., (1997).

Alternatively, the peptide inhibitor may be produced by recombinant technology methods as known in the art and as described, for example, in Sambrook et al., Molecular Cloning: A Laboratory Manual, Cold Springs Harbor laboratory, 2^{nd} ed., Cold Springs Harbor, N.Y. (1989), Martin, Robin, Protein Synthesis: Methods and Protocols, Humana Press, Totowa, N.J. (1998) and Current Protocols in Molecular Biology (Ausubel et al., eds.), John Wiley & Sons, which is regularly and periodically updated. For example, an expression vector may be used to produce the desired peptide inhibitor in an appropriate host cell and the product may then be isolated by known methods. The expression vector may include, for example, the nucleotide sequence encoding the desired peptide wherein the nucleotide sequence is operably linked to a promoter sequence.

As defined herein, a nucleotide sequence is “operably linked” to another nucleotide sequence when it is placed in a functional relationship with another nucleotide sequence. For example, if a coding sequence is operably linked to a promoter sequence, this generally means that the promoter may promote transcription of the coding sequence. Operably linked means that the DNA sequences being linked are typically contiguous and, where necessary to join two protein coding regions, contiguous and in reading frame. However, since enhancers may function when separated from the promoter by several kilobases and intronic sequences may be of variable length, some nucleotide sequences may be operably linked but not contiguous. Additionally, as defined herein, a nucleotide sequence is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide.

The β_{II}PKC inhibitor may be derived from the beta-2 (β_{II})-isozyme of PKC from any species, such as Homo sapiens (Genbank Accession No. Q14289; SEQ ID NO: 139), Rattus norvegicus (Genbank Accession No. P70600; SEQ ID NO: 140), or Mus musculus (Genbank Accession No. Q9QVP9; SEQ ID NO: 141). An exemplary β_{II}PKC is β_{II}V5-3, having the sequence QEVIRN (SEQ ID NO: 142; Stebbins, E. G. and Mochly-Rosen, D. (2001) J. Biol. Chem. 276:29644-50). The experiments performed in support of the present invention utilized a modified version of β_{II}V5-3 having an N-terminal cysteine (i.e., CQEVIRN; SEQ ID NO: 86) to aid in attachments of a conjugate (see below).

The δPKC inhibitor may be derived from the delta (δ)-isozyme of PKC from any species, such as Rattus norvegicus (Genbank Accession No. AAH76505; SEQ ID NO: 147) or Homo sapiens (Genbank Accession No. NP_{—}997704; SEQ ID NO: 148). Exemplary δPKC inhibitors include δV1-1, having a portion of the amino acid sequence of δPKC from Rattus norvegicus (i.e., SFNSYELGSL; SEQ ID NO:1); δV1-2, having the sequence ALTTDRGKTLV, representing amino acids 35 to 45 of rat δPKC found in Genbank Accession No. MH76505; SEQ ID NO: 2); δV1-5, having the sequence KAEFWLDLQPQAKV (SEQ ID NO: 3), representing amino acids 101 to 114 of rat δPKC found in Genbank Accession No. AAH76505); 6V5, having the sequence PFRPKVKSPRPYSNFDQEFLNEKARLSYSDKNLIDSMDQSAF AGFSFVNPKFEHLLED (SEQ ID NO:4), representing amino acids 569-626 of human δPKC found in Genbank Accession No. BAA01381, with the exception that amino acid 11 (aspartic acid) is substituted with a praline; and/or some combination of δV1-1, δV1-2, δV1-5 and δV5, including variants, derivatives, or consensus sequences, thereof. δV1-7, having the amino acid sequence MRAAEDPM (SEQ ID NO: 146), is an activator or δPKC.

The peptide inhibitors may include natural amino acids, such as the L-amino acids or non-natural amino acids, such as D-amino acids. The amino acids in the peptide may be linked by peptide bonds or, in modified peptides described herein, by non-peptide bonds.

A wide variety of modifications to the amide bonds which link amino acids may be made and are known in the art. Such modifications are discussed in general reviews, including in Freidinger, R. M. (2003) “Design and Synthesis of Novel Bioactive Peptides and Peptidomimetics” J. Med. Chem. 46:5553, and Ripka, A. S., Rich, D. H. (1998) “Peptidomimetic Design” Curr. Opin. Chem. Biol. 2:441. These modifications are designed to improve the properties of the peptide by increasing the potency of the peptide or by increasing the half-life of the peptide.

The potency of the peptide may be increased by restricting the conformational flexibility of the peptide. This may be achieved by, for example, including the placement of additional alkyl groups on the nitrogen or alpha-carbon of the amide bond, such as the peptoid strategy of Zuckerman et al, and the alpha modifications of, for example Goodman, M. et. al. ((1996) Pure Appl. Chem. 68:1303). The amide nitrogen and alpha carbon may be linked together to provide additional constraint (Scott et al. (2004) Org. Letts. 6:1629-1632).

The half-life of the peptide may be increased by introducing non-degradable moieties to the peptide chain. This may be achieved by, for example, replacement of the amide bond by a urea residue (Patil et al. (2003) J. Org. Chem. 68:7274-7280) or an aza-peptide link (Zega and Urleb (2002) Acta Chim. Slov. 49:649-662). Other examples of non-degradable moieties that may be introduced to the peptide chain include introduction of an additional carbon (“beta peptides”, Gellman, S. H. (1998) Acc. Chem. Res. 31:173) or ethene unit (Hagihara et al (1992) J. Am. Chem. Soc. 114:6568) to the chain, or the use of hydroxyethylene moieties (Patani, G. A. and Lavoie, E. J. (1996) Chem. Rev. 96:3147-3176) and are also well known in the art. Additionally, one or more amino acids may be replaced by an isosteric moiety such as, for example, the pyrrolinones of Hirschmann et al ((2000) J. Am. Chem. Soc. 122:11037), or tetrahydropyrans (Kulesza, A. et al. (2003) Org. Letts. 5:1163). The inhibitors may also be pegylated,

Although the peptides are described primarily with reference to amino acid sequences from Rattus norvegicus, it is understood that the peptides are not limited to the specific amino acid sequences set forth herein. Skilled artisans will recognize that, through the process of mutation and/or evolution, polypeptides of different lengths and having different constituents, e.g., with amino acid insertions, substitutions, deletions, and the like, may arise that are related to, or sufficiently similar to, a sequence set forth herein by virtue of amino acid sequence homology and advantageous functionality as described herein.

The peptide inhibitors described herein also encompass amino acid sequences similar to the amino acid sequences set forth herein that have at least about 50% identity thereto and function to inhibit tumor growth and/or angiogenesis. Preferably, the amino acid sequences of the peptide inhibitors encompassed in the invention have at least about 60% identity, further at least about 70% identity, preferably at least about 75% or 80% identity, more preferably at least about 85% or 90% identity, and further preferably at least about 95% identity, to the amino acid sequences set forth herein. Percent identity may be determined, for example, by comparing sequence information using the advanced BLAST computer program, including version 2.2.9, available from the National Institutes of Health. The BLAST program is based on the alignment method of Karlin and Altschul. Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997).

Conservative amino acid substitutions may be made in the amino acid sequences described herein to obtain derivatives of the peptides that may advantageously be utilized in the present invention. Conservative amino acid substitutions, as known in the art and as referred to herein, involve substituting amino acids in a protein with amino acids having similar side chains in terms of, for example, structure, size and/or chemical properties. For example, the amino acids within each of the following groups may be interchanged with other amino acids in the same group: amino acids having aliphatic side chains, including glycine, alanine, valine, leucine and isoleucine; amino acids having non-aromatic, hydroxyl-containing side chains, such as serine and threonine; amino acids having acidic side chains, such as aspartic acid and glutamic acid; amino acids having amide side chains, including glutamine and asparagine; basic amino acids, including lysine, arginine and histidine; amino acids having aromatic ring side chains, including phenylalanine, tyrosine and tryptophan; and amino acids having sulfur-containing side chains, including cysteine and methionine. Additionally, amino acids having acidic side chains, such as aspartic acid and glutamic acid, are considered interchangeable herein with amino acids having amide side chains, such as asparagine and glutamine.

Modifications to δV1-1 that are expected to inhibit δPKC, with a concomitant decrease in tumor volume, angiogenesis, HIF-1a expression, or VEGF expression, and/or increase the sensitivity of tumor cells to apoptosis, include the following changes to SEQ ID NO: 1 (shown in lower case and/or underlined): tFNSYELGSL (SEQ ID NO:5), aFNSYELGSL (SEQ ID NO:6), SFNSYELGtL (SEQ ID NO:7), including any combination of these three substitutions, such as tFNSYELGtL (SEQ ID NO:8). Other potential modifications include SyNSYELGSL (SEQ ID NO:9), SFNSfELGSL (SEQ ID NO:10), SNSYdLGSL (SEQ ID NO:11), SFNSYELpSL (SEQ ID NO:12).

Other possible modifications that are expected to produce a peptide that functions in the invention include changes of one or two L to I or V, such as SFNSYEiGSv (SEQ ID NO:13), SFNSYEvGSi (SEQ ID NO:14), SFNSYELGSv (SEQ ID NO:15), SFNSYELGSi (SEQ ID NO:16), SFNSYEiGSL (SEQ ID NO:17), SFNSYEvGSL (SEQ ID NO:18), aFNSYELGSL (SEQ ID NO:19), any combination of the above-described modifications, and other conservative amino acid substitutions described herein.

Fragments and modification of fragments of δV1-1 are also contemplated, including: YELGSL (SEQ ID NO:20), YdLGSL (SEQ ID NO:21), fdLGSL (SEQ ID NO:22), YdiGSL (SEQ ID NO:23), iGSL (SEQ ID NO:24), YdvGSL (SEQ ID NO:25), YdLpsL (SEQ ID NO:26), YdLgiL (SEQ ID NO:27), YdLGSi (SEQ ID NO:28), YdLGSv (SEQ ID NO:29), LGSL (SEQ ID NO:30), iGSL (SEQ ID NO:31), vGSL (SEQ ID NO:32), LpSL (SEQ ID NO:33), LGiL (SEQ ID NO:34), LGSi (SEQ ID NO:35), LGSv (SEQ ID NO:36).

Accordingly, the term “a δV1-1 peptide” as used herein further refers to a peptide identified by SEQ ID NO:1 and to a peptide having an amino acid sequence having the specified percent identity described herein to the amino acid sequence of SEQ ID NO:1, including but not limited to the peptides set forth in SEQ ID NOS:5-19, as well as fragments of any of these peptides that retain the ability to decrease tumor volume, angiogenesis, HIF-1a expression, or VEGF expression, and/or increase the sensitivity of tumor cells to apoptosis, as exemplified by but not limited to SEQ ID NOS:20-36.

Modifications to δV1-2 that are expected to result in effective inhibition of δPKC with a concomitant decrease in tumor volume, angiogenesis, HIF-1a expression, or VEGF expression, and/or increase the sensitivity of tumor cells to apoptosis, include the following changes to SEQ ID NO: 2 shown in lower case: ALsTDRGKTLV (SEQ ID NO:37), ALTsDRGKTLV (SEQ ID NO:38), ALTTDRGKsLV (SEQ ID NO:39), and any combination of these three substitutions, ALTTDRpKTLV (SEQ ID NO:40), ALTTDRGrTLV (SEQ ID NO:41), ALTTDkGKTLV (SEQ ID NO:42), ALTTDkGkTLV (SEQ ID NO:43), changes of one or two L to I, or V and changes of V to I, or L and any combination of the above. In particular, L and V can be substituted with V, L, I R and D, E can be substituted with N or Q. One skilled in the art would be aware of other conservative substitutions that may be made to achieve other derivatives of δV1-2 in light of the description herein.

Accordingly, the term “a δV1-2 peptide” as further used herein refers to a peptide identified by SEQ ID NO:2 and to a peptide having an amino acid sequence having the specified percent identity described herein to the amino acid sequence of SEQ ID NO:2, including but not limited to the peptides set forth in SEQ ID NOS:37-43, as well as fragments of any of these peptides that retain the ability to decrease tumor volume, angiogenesis, HIF-1a expression, or VEGF expression, and/or increase the sensitivity of tumor cells to apoptosis, as described.

Modifications to δV1-5 that are expected to result in effective inhibition of δPKC with a concomitant decrease in tumor volume, angiogenesis, HIF-1a expression, or VEGF expression, and/or increase the sensitivity of tumor cells to apoptosis, include the following changes to SEQ ID NO:3 shown in lower case: rAEFWLDLQPQAKV (SEQ ID NO:44); KAdFWLDLQPQAKV (SEQ ID NO:45); KAEFWLeLQPQAKV (SEQ ID NO:46), KAEFWLDLQPQArV (SEQ ID NO;47), KAEyWLDLQPQAKV (SEQ ID NO:48), KAEFWiDLQPQAKV (SEQ ID NO:49), KAEFWvDLQPQAKV (SEQ ID NO:50), KAEFWLDiQPQAKV (SEQ ID NO:51), KAEFWLDvQPQAKV (SEQ ID NO:52), KAEFWLDLnPQAKV (SEQ ID NO:53), KAEFWLDLQPnAKV (SEQ ID NO;54), KAEFWLDLQPQAKi (SEQ ID NO;55), KAEFWLDLQPQAKl (SEQ ID NO:56), KAEFWaDLQPQAKV (SEQ ID NO:57), KAEFWLDaQPQAKV (SEQ ID NO;58), and KAEFWLDLQPQAKa (SEQ ID NO:59).

Fragments of δV1-5 are also contemplated, including: KAEFWLD (SEQ ID NO:60), DLQPQAKV (SEQ ID NO:61), EFWLDLQP (SEQ ID NO:62), LDLQPQA (SEQ ID NO:63), LQPQAKV (SEQ ID NO:64), AEFWLDL (SEQ ID NO:65), and WLDLQPQ (SEQ ID NO:66).

Modifications to fragments of δV1-5 are also contemplated and include the modifications shown for the full-length fragments as well as other conservative amino acid substitutions described herein. The term “a δV1-5 peptide” as further used herein refers to SEQ ID NO:3 and to a peptide having an amino acid sequence having the specified percent identity described herein to an amino acid sequence of SEQ ID NO:3, as well as fragments thereof that retain the ability to decrease tumor volume, angiogenesis, HIF-1a expression, or VEGF expression, and/or increase the sensitivity of tumor cells to apoptosis, as described.

Modifications to δV5 that are expected to result in effective inhibition of δPKC with a concomitant decrease in tumor volume, angiogenesis, HIF-1a expression, or VEGF expression, and/or increase the sensitivity of tumor cells to apoptosis, include making one or more conservative amino acid substitutions, including substituting: R at position 3 with Q; S at position 8 with T; F at position 15 with W; V at position 6 with L and D at position 30 with E; K at position 31 with R; and E at position 53 with D, and various combinations of these modifications and other modifications that can be made by the skilled artisan in light of the description herein.

Fragments of δV5 are also contemplated, and include, for example, the following: SPRPYSNF (SEQ ID NO:67), RPYSNFDQ (SEQ ID NO:68), SNFDQEFL (SEQ ID NO:69), DQEFLNEK (SEQ ID NO:70), FLNEKARL (SEQ ID NO:71), LIDSMDQS (SEQ ID NO:72), SMDQSAFA (SEQ ID NO:73), DQSAFAGF (SEQ ID NO:74), FVNPKFEH (SEQ ID NO:75), KFEHLLED (SEQ ID NO:76), NEKARLSY (SEQ ID NO:77), RLSYSDKN (SEQ ID NO:78), SYSDKNLI (SEQ ID NO:79), DKNLIDSM (SEQ ID NO:80), PFRPKVKS (SEQ ID NO: 81), RPKVKSPR (SEQ ID NO:82), and VKSPRPYS (SEQ ID NO:83).

Modifications to fragments of δV5 are also contemplated and include the modifications shown for the full-length fragments as well as other conservative amino acid substitutions described herein. The term “a δV5 peptide” as further used herein refers to SEQ ID NO: 4 and to a peptide having an amino acid sequence having the specified percent identity described herein to an amino acid sequence of SEQ ID NO: 4, as well as fragments thereof that retain the ability to decrease tumor volume, angiogenesis, HIF-1a expression, or VEGF expression, and/or increase the sensitivity of tumor cells to apoptosis, as described.

Modifications to the β_{II}V-5-3 peptide that are expected to result in effective reduction in tumors size, the levels of VEGF and/or angiogenesis in tumor tissues, or increases the level of apoptosis in tumor cells, include the following changes to SEQ ID NO:86 (shown in lower case): CnEVIRN (SEQ ID NO:87), CQdVIRN (SEQ ID NO:88), CQEgIRN (SEQ ID NO:89), CQEaIRN (SEQ ID NO:90), CQElIRN (SEQ ID NO:91), CQEiIRN (SEQ ID NO:92), CQEVgRN (SEQ ID NO:93), CQEVaRN (SEQ ID NO:94), CQEVvRN (SEQ ID NO:95), CQElIRN (SEQ ID NO:96), CQEVIkN (SEQ ID NO:97), CQEVIhN (SEQ ID NO:98), CQEVIRq (SEQ ID NO:99) and QEVIRN (SEQ ID NO: 100).

Suitable β_{II}V-5-3 peptide may also comprise more than one substitution, including but not limited to CndVIRN (SEQ ID NO:101), CnEVgIRN (SEQ ID NO:102), CnEVaIRN (SEQ ID NO:103), CnEVlIRN (SEQ ID NO:104), CnEVvIRN (SEQ ID NO:105), CnEViIRN (SEQ ID NO:106), CnEVIkN (SEQ ID NO:107), CnEVIhN (SEQ ID NO:108), CnEVIRq (SEQ ID NO:109), CQdVgIRN (SEQ ID NO:110), CQdVaIRN (SEQ ID NO:111), CQdVlIRN (SEQ ID NO:112), CQdVvIRN (SEQ ID NO:113), CQdViIRN (SEQ ID NO:114), CQdVIkN (SEQ ID NO:115), CQdVIhN (SEQ ID NO:116), CQdVIRq (SEQ ID NO:117), CQEggRN (SEQ ID NO:118), CQEgaRN (SEQ ID NO:119), CQEgvRN (SEQ ID NO:120), CQEglRN (SEQ ID NO:121), CQEagRN (SEQ ID NO:122), CQEaaRN (SEQ ID NO:123), CQEavRN (SEQ ID NO:124), CQEalRN (SEQ ID NO:125), CQEigRN (SEQ ID NO:126), CQEiaRN (SEQ ID NO:127), CQEivRN (SEQ ID NO:149), CQEilRN (SEQ ID NO:128), CQElgRN (SEQ ID NO:129), CQElaRN (SEQ ID NO:130), CQElvRN (SEQ ID NO:131), CQEllRN (SEQ ID NO:132), CQElgRN (SEQ ID NO:133), CQElaRN (SEQ ID NO:134), CQElvRN (SEQ ID NO:135), CQEVvkN (SEQ ID NO:136), CQEVikN (SEQ ID NO:137), and CQEVlkq (SEQ ID NO:138), other peptide variants, fragments, and/or derivatives are expected to produce acceptable results.

The terms “β_{II}V5-3 peptide” is used to refer generally to peptides having the features described herein, not limited to the peptide of SEQ ID NO: 86. Also included within this definition, and in the scope of the invention, are variants of the peptides which function in inhibiting tumor growth. Examples of these peptides are described above.

Other suitable molecules or compounds, including small molecules and peptidomimetic compounds that act as inhibitors of β_{II}PKC or δPKC, may be identified by methods known to the art. For example, such molecules may be identified by their ability to inhibit translocation of β_{II}PKC or δPKC to its subcellular location. Such assays may utilize, for example, fluorescently-labeled enzyme and fluorescent microscopy to determine whether a particular compound or agent may aid in the cellular translocation of β_{II}PKC or δPKC. Such assays are described, for example, in Schechtman, D. et al. (2004) J. Biol. Chem. 279:1583140, and include use of selected antibodies. Other assays to measure cellular translocation include Western blot analysis as described in Dorn, G. W. et al. (1999) Proc. Natl. Acad. Sci. U.S.A. 96:12798-12803 and Johnson, J. A. and Mochly-Rosen, D. (1995) Circ Res. 76:654-63.

The β_{II}PKC or δPKC inhibitors may be modified by being part of a fusion protein. The fusion protein may include a protein or peptide that functions to increase the cellular uptake of the peptide inhibitors, has another desired biological effect, such as a therapeutic effect, or may have both of these functions. For example, it may be desirable to conjugate, or otherwise attach, the δV1-1 peptide, the β_{II} V-5-3 peptide, or other peptides described herein, to a cytokine or other protein that elicits a desired biological response. The fusion protein may be produced by methods known in the art. For example, the inhibitor peptide may be bound to a carrier peptide, such as a cell permeable carrier peptide, or other peptide described herein via cross-linking wherein both peptides of the fusion protein retain their activity. As a further example, the peptides may be linked or otherwise conjugated to each other by an amide bond from the C-terminal of one peptide to the N-terminal of the other peptide. The linkage between the inhibitor peptide and the other member of the fusion protein may be non-cleavable or cleavable with, for example, an esterase or peptidase.

Furthermore, in other forms of the invention, the carrier protein, such as a cell permeable carrier peptide, or other peptide that may increase cellular uptake of the peptide inhibitor may be, for example, a Drosophila Antennapedia homeodomain-derived sequence which is set forth in SEQ ID NO:84 (CRQIKIWFQNRRMKWKK), and may be attached to the inhibitor by cross-linking via an N-terminal Cys-Cys bond as discussed in Theodore, L., et al. (1995) J. Neurosci. 15:7158-7167 and Johnson, J. A., et al. (1996) Circ. Res 79:1086. Alternatively, the inhibitor may be modified by a Transactivating Regulatory Protein (Tat)-derived transport polypeptide (such as from amino acids 47-57 of Tat shown in SEQ ID NO:85; YGRKKRRQRRR) from the Human Immunodeficiency Virus, Type 1, as described in Vives, et al. (1997) J. Biol. Chem., 272:16010-17; U.S. Pat. No. 5,804,604; and Genbank Accession No. MT48070; or with polyarginine as described in Mitchell, et al. (2000) J. Peptide Res. 56:318-25 and Rothbard, et al. (2000) Nature Med. 6:1253-57. Examples of Tat-conjugate peptides are provided in Example 2. The inhibitors may be modified by other methods known to the skilled artisan in order to increase the cellular uptake of the inhibitors.

While the present invention has largely been described in terms of polypeptides/peptide inhibitors, the invention includes administering to an animal in need of such treatment a polynucleotide encoding any of the polypeptide/peptide inhibitors described herein. Polynucleotide encoding peptide inhibitors include gene therapy vectors based on, e.g., adenovirus, adeno-associated virus, retroviruses (including lentiviruses), pox virus, herpesvirus, single-stranded RNA viruses (e.g., alphavirus, flavivirus, and poliovirus), etc. Polynucleotide encoding polypeptides/peptide inhibitors further include naked DNA or plasmids operably linked to a suitable promoter sequence and suitable of directing the expression of any of the polypeptides/peptides described, herein.

E. Administration and Dosing of PKC Inhibitors

An osmotic pump was used to deliver the β_{II}PKC or δPKC inhibitors to experimental animals (see above and the Examples). The osmotic pump allowed a continuous and consistent dosage of β_{II}PKC or δPKC inhibitors to be delivered to animals with minimal handling. Nonetheless, osmotic pumps are generally not the preferred method for delivering β_{II}PKC or δPKC inhibitors.

The inhibitors may be administered in various conventional forms. For example, the inhibitors may be administered in tablet form for sublingual administration, in a solution or emulsion. The inhibitors may also be mixed with a pharmaceutically-acceptable carrier or vehicle. The vehicle may be a liquid, suitable, for example, for parenteral administration, including water, saline or other aqueous solution, or may be an oil or an aerosol. The vehicle may be selected for intravenous or intraarterial administration, and may include a sterile aqueous or non-aqueous solution that may include preservatives, bacteriostats, buffers and antioxidants known to the art. In the aerosol form, the inhibitor may be used as a powder, with properties including particle size, morphology and surface energy known to the art for optimal dispersability. In tablet form, a solid vehicle may include, for example, lactose, starch, carboxymethyl cellulose, dextrin, calcium phosphate, calcium carbonate, synthetic or natural calcium allocate, magnesium oxide, dry aluminum hydroxide, magnesium stearate, sodium bicarbonate, dry yeast or a combination thereof. The tablet preferably includes one or more agents which aid in oral dissolution. The inhibitors may also be administered in forms in which other similar drugs known in the art are administered, including patches, a bolus, time release formulations, and the like.

The inhibitors described herein may be administered for prolonged periods of time without causing desensitization of the patient to the inhibitor. That is, the inhibitors can be administered multiple times, or after a prolonged period of time including one, two or three or more days; one two, or three or more weeks or several months to a patient and will continue to cause an increase in the flow of blood in the respective blood vessel.

The inhibitors may be administered to a patient by a variety of routes. For example, the inhibitors may be administered parenterally, including intraperitoneally; intravenously; intraarterially; subcutaneously, or intramuscularly. The inhibitors may also be administered via a mucosal surface, including rectally, and intravaginally; intranasally; by inhalation, either orally or intranasally; orally, including sublingually; intraocularly and transdermally. Combinations of these routes of administration are also envisioned.

A therapeutically effective amount of the inhibitor is provided. As used herein, a therapeutically effective amount of the inhibitor is the quantity of the inhibitor required to decrease tumor proliferation or growth, decrease morbidity or mortality associated with one or more tumors, or improve the quality of life for animals having tumors. The description provides guidance for selecting β_{II}PKC or δPKC inhibitors, assays for measuring tumor growth, tumor cell proliferation, and the rate of apoptosis in tumor cells, and exemplary dosages and dosing schedules that can be extrapolated to a variety of animals. Preferred PKC inhibitors demonstrate similar biological activities as those inhibitors described, e.g., β_{II}V5-3 and δV1-1, using the assays provided.

The skilled artisan will be able to determine the optimum dosage. Generally, the amount of inhibitor utilized may be, for example, about 0.0005 mg/kg body weight to about 50 mg/kg body weight, but is preferably about 0.05 mg/kg to about 0.5 mg/kg. The exemplary concentration of the inhibitors and activators used herein are from 3 mM to 30 mM but concentrations from below about 0.01 mM to above about 100 mM (or to saturation) are expected to provide acceptable results.

The amount of inhibitor is preferably sufficient to decrease tumor growth, decreases cell proliferation, or decrease morbidity/mortality by at least about 5%, by at least about 10%, preferably at least about 25%, further at least about 50%, more preferably at least about 75% and further at least about 100% compared to the clinical condition prior to treatment or compared to untreated animals.

The patient to be treated is typically one in need of such treatment, including a patient having a prostate tumor, or susceptible to developing a prostate tumor. The tumor may be androgen-dependent or androgen-independent, and may be a primary tumor or secondary tumor resulting from metastasis. The patient is typically a vertebrate and preferably a mammal, including a human. Other animals which may be treated include farm animals (such as horse, sheep, cattle, and pigs); pets (such as cats, dogs); rodents, mice, rats, gerbils, hamsters, and guinea pigs; members of the order Lagomorpha (including rabbits and hares); and any other mammal that may benefit from such treatment.

While the β_{II}PKC and δPKC inhibitors of the invention have largely been discussed separately, one skilled in the art will recognize that combination treatment (i.e., using β_{II}PKC and δPKC inhibitors) may provide additional therapeutic benefit. In addition, the β_{II}PKC and δPKC inhibitors of the invention may be combined with conventional procedures and drugs for treating prostate tumors (e.g., chemotherapy, radiation therapy, surgery (including orchiectomy), treatment with luteinizing hormone-releasing hormone (LH-RH) agonists, and anti-androgen therapy).

F. Compositions and Kits

The present invention further provides novel polypeptide/peptide and/or peptimimetic inhibitors of β_{II}PKC and δPKC, some of which are identified herein. These compositions may be provided as a formulation in combination with a suitable pharmaceutical carrier, which encompasses liquid formulations, tablets, capsules, films, etc. The β_{II}IPKC and/or δPKC inhibitors may also be supplied in lyophilized form.

Such compositions may be a component of a kit of parts (i.e., kit) for treating prostate tumors. In addition to a PKR inhibitor composition, such kits may include administration and dosing instructions, instructions for identifying patients in need of treatment, and instructions for monitoring a patients' response to PKR inhibitor therapy. Where the PKR inhibitor is administered via a pump (as in the animal studies described, herein), the kit may comprise a pump suitable for delivering PKR inhibitors.

The following examples are provided to illustrate the invention. Additional embodiments of the invention will apparent to one skilled in the art without departing from the scope of the invention.

### EXAMPLES

### Example 1

### PKC and TAT₄₇₋₅₇ Peptides

The PKC peptides and TAT₄₇₋₅₇ were synthesized and conjugated via a Cys S—S bond as described previously (Chen, et al. (2001) Proc. Natl. Acad. Sci. USA 25:11114-19 and Inagaki, et al. (2003) Circulation 11:2304-07).

### Example 2

### Administration of Peptide Inhibitors and Activators

Male nude mice were subcutaneously injected with human prostate cancer cells (PC3) at six weeks of age. After one week, the animals were implanted with an ALZEJ® (Alza Corporation, Mountain View, Calif.) osmotic pump for delivery of a control saline solution, a control peptide of TAT (residues 47-57, YGRKKRRQRRR SEQ ID NO:85), or an inhibitor or activator of PKC (e.g., δV1-1 attached to TAT (YGRKKRRQRRR-CC-SFNSYELGSL; SEQ ID NO: 143), δV1-7 attached to TAT (YGRKKRRQRRR-CC-MRAAEDPM; SEQ ID NO: 144), or β_{II}V5-3 attached to TAT (YGRKKRRQRRR-CC-QEVIRN; SEQ ID NO: 145). The rate of administration was 0.5 μl/hr, unless otherwise noted. Typical inhibitor or activator concentrations were 3-30 mM. In some cases, a lower concentration was administered initially (e.g., 3 mM) followed by a higher concentration (e.g., 30 mM) in the later weeks of treatment.

Tumor volumes were measured periodically (e.g., weekly). The mice were typically sacrificed after 5 weeks of treatment. Deuterated water was given to the animals about one week prior to sacrifice to facilitate the measurement of cell proliferation. Angiogenesis and tumor cell proliferation were measured at six weeks by deuterium analyses using gas chromatography-mass spectrometry (GC-MS). Ribose derivatives extracted from DNA that incorporated deuterium during cell division can be identified by GC-MS and can be quantitated over total ribose from all DNA. This measurement allows the calculation of “newly synthesized DNA” during the deuterated water administration (i.e., pulse), from which the fractional turnover rate can be calculated using an exponential equation. Levels of tumor cell markers, angiogenesis-related polypeptides, and apoptosis-related proteins were evaluated by Western blot and immunohistochemistry. The results of experiments using these methods are shown in the Figures.

### Example 3

### Immunoblot Analysis and Quantitation of Soluble and Particulate PKC

Immunoblot analysis is well-known in the art and described, for example, in Sambrook et al., Molecular Cloning: A Laboratory Manual, Cold Springs Harbor laboratory, 2^{nd} ed., Cold Springs Harbor, N.Y. (1989) and Current Protocols in Molecular Biology (Ausubel et al., eds.), John Wiley & Sons, which is regularly and periodically updated.

In one particular protocol, Western blot analyses of normal prostate cells or prostate tumor or grown on 100 mm glass dishes were carried out as previously described (Liu, Y., et al., 1995). Following treatment, medium from one plate was removed, and cells were washed twice with ice-cold phosphate-buffered saline (PBS). 1.5 ml of chilled homogenization buffer consisting of 10 mM Tris-HCl pH 7.4, 1 mM EDTA, 1 mM EGTA, 0.25 M sucrose, and 20 mg/ml each of phenylmethylsulfonyl fluoride, soybean trypsin inhibitor, leupeptin, and aprotinin was added to each dish. Cells were scraped from the plates and triturated 3 times with a tuberculin syringe attached to a 22-gauge needle. The resulting lysates were centrifuged at 4° for 30 minutes at 100,000.times g in a Beckman Ti 100.3 rotor (Beckman Instruments, Columbia, Md.). Supernatants were concentrated to a volume of 250 ml with a Centricon 30 filtration unit (Amicon, Beverly, Mass.). Pellets were resuspended in 250 ml of homogenization buffer with a tuberculin syringe attached to a 22-gauge needle. Soluble and particulate fractions were then subjected to 12% SDS-PAGE and transferred to nitrocellulose sheets.

The antibodies used to probe the blots included the following:

| Antibody | Source |
|---|---|
| anti-β_{I}PKC, anti-β_{II}PKC, | Santa Cruz Biotechnology |
| anti-αPKC, anti-εPKC, | |
| anti zetaPKC, anti-δPKC, | |
| anti-Gα1, anti-Ki67 | |
| anti-VEGF | R&D Diagnostics (ELISA kit) |
| anti-HIF-1a | Bethyl Laboratories, Inc., A300-286A |
| anti-CD31 | Pharmingen |
| anti-cleaved caspase 3 | Signal Transduction |

### Example 4

### Peptide Activation of PKC Assayed by Substrate Phosphorylation

Activation of .epsilon-PKC by peptide epsilon-V1-7 was measured by phosphorylation of one of its substrates, calsequestrin. The epsilon-V1-7 peptide (10 mM) was incubated with epsilon-PKC (about 10 nM) for 15 minutes at room temperature in overlay buffer (50 mM Tris-HCl pH 7.5 containing 0.1% bovine serum albumin (BSA), 5 mg/ml leupeptin, 10 mg/ml soybean trypsin inhibitor (SBTI), 0.1% polyethylene glycol (PEG), 0.2M NaCl, 0.1 mM CaCl.sub.2 and 12 mM .beta.-mercaptoethanol). Calsequestrin (0.2 mg/ml) was then added to the mixture along with 20 mM Tris-HCl pH 7.5 containing MgCl.sub.2 (20 mM), 2-meracptoethanol (12 mM), ATP (20 mM) and [γ³²P]ATP (5 mCi/ml). In some experiments (indicated), the PKC activators DG (1.2.mu.g/ml) and/or PS (50 pg/ml) were also added. The mixture was incubated for 15 minutes at room temperature and the reaction stopped by addition of sample buffer. The samples were then boiled for 10 minutes and loaded onto 10% SDS-PAGE minigel. The gel was fixed with 50% methanol and 10% acetic acid for 1 hour and calsequestrin phosphorylation was determined by autoradiography.

### Example 5

### Inhibition of Delta-PKC Translocation

### A. Peptide Preparation

δV5 PKC peptides are synthesized and purified. The peptides are modified with a carrier peptide by cross-linking via an N-terminal Cys-Cys bond to the Drosophila Antennapedia homeodomain (Théodore, L., et al. J. Neurosci., 15:7158 (1995); Johnson, J. A., et al., Circ. Res., 79:1086 (1996)) or a Tat-derived peptide.

B. Peptide Delivery into Cells

The peptides are introduced into cells at an applied concentration of 500 nM in the presence and absence of phorbol 12-myristate 13-acetate (PMA) at concentrations of 3 nm and 10 nm, respectively, for 10-20 minutes. In a third set of cells, the peptides are applied at a concentration of 500 nM in the presence and absence of 500 nM δRACK.

Translocation of the δPKC isozyme is assessed by using δPKC isozyme-specific antibodies in Western blot analysis (Santa Cruz Biotechnology). Western blot analysis of cystosolic and particulate fractions of treated cells is carried out as described previously (Johnson, J. A., et al., Circ. Res. 76:654 (1995)). Subcellular localization of δPKC isozymes is assessed by chemiluminescence of blots probed with anti-δPKC, anti-.αPKC and anti-epsilon-PKC antibodies. Amounts of δPKC isozymes in each fraction are quantitated using a scanner and translocation is expressed as the amount of isozymes in the particulate fraction over the amount of isozymes in non-treated cells. Changes in translocation of δPKC isozyme are also determined by immunofluoresence study of treated and fixed cells (Gray, M. O. et al., J. Biol. Chem., 272:30945-3095 (1997)). Translocation is determined by counting over 100 cells/treatment in a blinded fashion.

### Example 6

### Identification of Compounds that Mimic the Activity of PKC Isozymes

A competitive binding screening assay can be used to identify compounds that mimic the activity of a PKC isozyme by adding a test compound and a detectably labeled peptide of the invention to mammalian cells, tissue, or the suitable RACK under conditions that allow binding of the peptide and comparing the results against binding of the labeled peptide (without test compound) to the cell, tissue or RACK. Compounds that mimic the activity of the peptide can compete with the peptide for binding to the cell, tissue or RACK. Consequently, a smaller amount of RACK-bound labeled peptide (or a larger amount of RACK-unbound labeled peptide) will be measured when the test compound mimics the activity of the peptide by binding to the receptor (as compared to the amounts of free and RACK-bound labeled peptide when a test compound does not mimic the activity of the peptide, does not bind to the receptor, or does so with less affinity).

In general, identification of compounds that mimic the activity of PKC isozymes are identified by measuring the ability of a test compound to inhibit, enhance, or modulate the activity of the corresponding PKC isozyme. The activity of the PKC isozyme in a selected assay is measured in the presence and absence of the test compound. The assay can be a competitive binding assay (e.g., as described above) or a cellular assay the monitors modulation of a second messenger production, changes in cellular metabolism, or effects on enzymatic activity. Compounds identified as mimicking or modulating the activity of the PKC isozyme are then tested for therapeutic activity using a corresponding in vivo disease model.

What is claimed is:

1. A treatment method, comprising
administering an inhibitor of delta protein kinase C (δPKC) or an inhibitor of beta-1 protein kinase C (β_{II}PKC) in an amount effective to decrease the rate of growth of a solid tumor.

2. A treatment method, comprising
administering an inhibitor of delta protein kinase C or an inhibitor of beta-II protein kinase C (β_{II}PKC) in an amount effective to inhibit tumor angiogenesis.

3. The method of claim 1 or claim 2, wherein the inhibitor of δPKC is a peptide.

4. The method of claim 3, wherein said peptide is selected from the first variable region of δPKC.

5. The method of claim 3, wherein said peptide is a peptide having between about 5 and 15 contiguous residues from the first variable region of δPKC.

6. The method of claim 3, wherein said peptide has at least about 50% sequence identity with a conserved set of between about 5 and 15 contiguous residues from the first variable region of δPKC.

7. The method of claim 4, wherein the peptide has at least about 80% sequence identity with SFNSYELGSL (SEQ ID NO:1).

8. The method of claim 7, wherein said peptide is modified to include a carrier molecule.

9. The method of claim 8, wherein said peptide is modified to include a terminal Cys residue.

10. The method of claim 8, wherein said peptide is modified to include an N-terminal Cys residue.

11. The method of claim 8, wherein said carrier molecule is selected from a Drosophila Antennapedia homeodomain-derived sequence (CRQIKIWFQNRRMKWKK, SEQ ID NO: 84), a Transactivating Regulatory Protein (Tat)-derived transport polypeptide from the Human Immunodeficiency Virus, Type 1 (YGRKKRRQRRR, SEQ ID NO: 85), or a polyarginine.

12. The method of claim 1 or claim 2, wherein the inhibitor of β_{II}PKC is a peptide.

13. The method of claim 12, wherein said peptide is selected from the fifth variable region of β_{II}PKC.

14. The method of claim 12, wherein said peptide is a peptide having between about 5 and 15 contiguous residues from the fifth variable region of β_{II}PKC.

15. The method of claim 12, wherein said peptide has at least about 50% sequence identity with a conserved set of between about 5 and 15 contiguous residues from the fifth variable region of β_{II}PKC.

16. The method of claim 13, wherein the peptide has at least about 80% sequence identity with QEVIRN (SEQ ID NO: 142).

17. The method of claim 16, wherein said peptide is modified to include a carrier molecule.

18. The method of claim 17, wherein said peptide is modified to include a terminal Cys residue.

19. The method of claim 18, wherein said peptide is modified to include an N-terminal Cys residue.

20. The method of claim 17, wherein said carrier molecule is selected from a Drosophila Antennapedia homeodomain-derived sequence (CRQIKIWFQNRRMKWKK, SEQ ID NO: 84), a Transactivating Regulatory Protein (Tat)-derived transport polypeptide from the Human Immunodeficiency Virus, Type 1 (YGRKKRRQRRR, SEQ ID NO: 85), or a polyarginine.

21. The method of claim 1, wherein the solid tumor is a tumor of the prostate.

22. The method of claim 2, wherein the tumor angiogenesis is associated with a tumor or a tumor cell in the prostate.

23. The method of claim 2, wherein the tumor angiogenesis is associated with a metastasized tumor cell.
